(19) United States
(12) Patent Application Publication
(10) Pub. No.: US 2022/0047718 A1
(43) Pub. Date: Feb. 17, 2022

(54) Nanoparticle-Based Therapy of Inflammatory Disorders

(71) Applicant: Midatech Ltd, Cardiff, South Glamorgan (GB)

(72) Inventors: Martina McAteer, Cardiff (GB); Tom Coulter, Cardiff (GB); Yao Ding, Cardiff (GB); John Porter, Cardiff (GB); Onur Boyman, Zürich (CH); Antonios G.A. Kolios, Zürich (CH); Alaz Özcan, Zürich (CH)

(21) Appl. No.: 17/413,495

(22) PCT Filed: Dec. 13, 2019
(86) PCT No.: PCT/EP2019/085206

§ 371 Date: Jun. 11, 2021

(30) Foreign Application Priority Data
Dec. 14, 2018 (GB) 1820471.9

(51) Int. Cl. A61K 47/69; A61K 47/54; A61K 9/00; A61P 37/06; A61K 31/519

(52) U.S. Cl. CPC A61K 47/6923; A61K 47/6929; A61K 47/549; A61K 47/6903; B82Y 5/00; A61K 9/0014; A61P 37/06; A61K 31/519; A61K 9/0019

(57) ABSTRACT

The present invention provides a nanoparticle comprising: a core comprising a metal and/or a semiconductor; and a plurality of ligands covalently linked to the core, wherein said ligands comprise: (i) at least one dilution ligand comprising a carbohydrate, glutathione or a polyethyleneglycol moiety; and (ii) a ligand of the formula MTX-L-, wherein MTX-L-represents methotrexate coupled to said core via a linker L. Also provided are pharmaceutical compositions of the nanoparticle, including gel formulations, and medical uses of the nanoparticle and pharmaceutical compositions, including for the treatment of an inflammatory or autoimmune disorder, such as psoriasis.

This application claims priority from GB1820471.9 filed 14 Dec. 2018, the contents and elements of which are herein incorporated by reference for all purposes.

### FIELD OF THE INVENTION

The present invention relates to nanoparticles as vehicles for delivery of active agents to specific tissue types or locations, particularly for use in medicine, and includes methods for treatment of inflammatory and/or autoimmune disorders, particularly skin disorders such as psoriasis. Pharmaceutical compositions, including topical gel formulations, and methods for their use are also disclosed.

### BACKGROUND TO THE INVENTION

The present invention is directed at compositions and products, and methods of making and administering such compositions and products, including for the treatment of mammals and particularly humans.

Psoriasis is a chronic multifactorial inflammatory skin disease that affects over 100 million people worldwide (˜2% of the general population). Although the exact aetiology of the disease is not known, it is generally considered to be an autoimmune disease where the stimulation of the immune system leads to hyperproliferation of epidermal keratinocytes and cutaneous inflammation.

Among the several forms, psoriasis vulgaris or plaque psoriasis is the most common affecting 80% of individuals and is characterized by red raised skin (plaques) and silvery white scales on the skin. The severity of the disease varies from mild (<3% of body), moderate (3-10% of body) to severe (>10% of body) depending on the percent of the total body area affected by psoriasis. A majority (75-80%) of the patients suffer from mild to moderate psoriasis.

Topical treatments are usually the first line of treatment for psoriasis to slow down or normalize excessive cell proliferation and reduce inflammation. Topical agents including Vitamin D analogues, corticosteroids, retinoids, or UV phototherapy are used for mild psoriasis, while patients with moderate to severe psoriasis are treated with systemic agents including methotrexate, ciclosporin, hydroxycarbamide, fumarates such as dimethyl fumarate, and retinoids, or biological agents (e.g. anti-TNF antibodies (e.g. infliximab), anti-IL-17 antibodies (e.g. ixekizumab), or anti-IL-23 antibodies (e.g. guselkumab)). However, these treatment options are, in many respects, sub-optimal. Systemic agents may be associated with severe side effects such as toxicity, while long-term UV phototherapy may be associated with carcinogenicity. For the majority of patients, particularly those having mild to moderate psoriasis, topical therapy is the preferred treatment of choice. However, the current topical agents are sub-optimal due to poor skin penetration and side effects associated with their use (e.g., skin thinning and skin irritation). Given these challenges, there is a strong unmet clinical need to develop a safe and effective topical therapy for psoriasis to achieve a high local drug concentration in the skin and reduce or eliminate the side effects associated with existing therapeutic options.

Methotrexate (MTX), a folic acid analogue is an anti-proliferative and anti-inflammatory agent. It inhibits DNA synthesis by irreversibly blocking the action of dihydrofolate reductase. It is currently administered for psoriasis by oral route or injection. However, its systemic use by physicians is limited due to the severe side effects including bone marrow toxicity, decreased white blood cell and platelet counts, liver damage, diarrhoea, gastric irritation, and ulcerative stomatitis. Given that MTX has an inhibitory effect on epidermal mitosis, topical application would be an attractive treatment option for psoriasis. However, the attempts to develop a topical MTX formulation for psoriasis have met with limited clinical success mainly due to the failure to reach sufficiently high drug concentration in the skin for an adequate period of time. The skin penetration of MTX is severely limited.

Various approaches have been investigated to improve the skin penetration of MTX including use of chemical enhancers, physical methods such as iontophoresis and lipid carriers. These approaches have achieved limited success, however, due to skin irritation issues, low drug loading, and limited skin penetration.

WO2014/028608 describes a method of treating skin disorders using nanoscale delivery devices and transdermal enhancing compositions. In particular, a zein shell-core nanoparticle encapsulating MTX was found to exhibit higher skin penetration than free MTX solution.

In the field of cancer treatment and imaging, gold nanoparticles loaded with MTX have been described. For example, US2015/0231077 describes gold nanoparticles passivated with amine-containing molecules, including MTX. Chen et al., Molecular Pharmaceutics, 2007, Vol. 4, No. 5, pp. 713-722, describes MTX adsorbed to 13 nm colloidal gold nanoparticles (see scheme 1) and subsequent assessment of the cytotoxic effect of MTX-AuNP on various cancer cells. Tran et al., Biochemical Engineering Journal, 2013, Vol. 78, pp. 175-180, describes fabrication of methotrexate-conjugated gold nanoparticles via a one-pot synthesis, and subsequent in vitro testing of MTX-AuNPs against cancer cells.

Bessar et al., Colloids and Surfaces B: Biointerfaces, 2016, Vol. 141, pp. 141-147, describes non-covalent loading of MTX onto water-soluble gold nanoparticles functionalised with sodium 3-mercapto-1-propansulfonate (Au-3MPS) and proposes that Au-3MPS@MTX could be suitable as a topical therapy in psoriasis patients. The loading efficiency of MTX on Au-3MPS was assessed in the range of 70-80%, with a fast release (80% in one hour). The Au-3MPS@MTX was used topically on normal skin of C57BL/6 mice in order to trace absorption behaviour. Skin penetration of Au-3MPS@MTX was found to be greater when compared to MTX alone. Penetration of psoriatic skin was not investigated, nor was efficacy of Au-3MPS@MTX as a psoriasis treatment assessed. Fratoddi et al., Nanomedicine: Nanotechnology, Biology and Medicine, 2019, Vol. 17, pp. 276-286 describe effects of topical Au-3MPS@MTX in cutaneous inflammatory mouse model.

There remains an unmet need for further nanoparticle delivery systems and for methods of treatment of psoriasis. In particular, nanoparticles that exhibit improved MTX loading and pharmaceutical compositions thereof, which exhibit efficacy in models of psoriasis remain an unmet need. The present invention seeks to provide solutions to these needs and provides further related advantages.

### BRIEF DESCRIPTION OF THE INVENTION

Broadly, the present invention relates to nanoparticles and compositions thereof, including gel-based pharmaceutical compositions for topical administration, that find use in the treatment of inflammatory or autoimmune disorders, such as psoriasis. The present inventors have surprisingly found that methotrexate-loaded nanoparticles, as described further herein, exhibit efficacy in vivo against models of psoriasis, reducing skin thickening and inflammation and even inhibiting onset of psoriasis. Significantly, the examples described herein demonstrate synergy between the gold nanoparticle and methotrexate. A gel formulated with GNPs alone (i.e. without MTX) caused a modest but significant reduction of ear thickness (FIG. 4c). The MTX-GNPs of the invention as defined herein were found to exhibit greater than additive efficacy on the skin inflammation models.

In a first aspect the present invention provides nanoparticle comprising:
a core comprising a metal and/or a semiconductor; and
a plurality of ligands covalently linked to the core, wherein said ligands comprise:
(i) at least one dilution ligand comprising a carbohydrate, glutathione or an ethylene glycol-containing moiety (e.g. an oligoethylene glycol or a (poly)ethylene glycol); and
(ii) a ligand of the formula MTX-L-, wherein MTX-L- represents methotrexate coupled to said core via a linker L.

Linker L may include a terminal group, such as a thiol group, that is covalently bound to the core. Alternatively, the linker L may be indirectly attached to the core via a spacer that is in turn covalently bound to the core.

In some embodiments linker L comprises a linear chain of 2 to 200 (e.g. 2 to 100, or 5 to 50) atoms in length between the methotrexate and the core. The linear chain may optionally be substituted, comprise side chains and/or be branched. The length of the linear chain is the number of atoms in the longest length between the methotrexate attachment site and the core.

In some embodiments L comprises a group —(CH₂)ₙ— and/or —(OCH₂CH₂)ₘ—, wherein n and m are independently 1. For example, L may comprise —(OCH₂CH₂)ₘ—, where m is a number in the range 5 to 20.

In some embodiments L is of the formula: L₁-Z-L₂ wherein L₁ comprises a first linker portion comprising a C2-C12 glycol and/or C1-C12 or C2-C12 alkyl chain, L₂ comprises a second linker portion comprising a C2-C12 glycol and/or C1-C12 or C2-C12 alkyl chain, wherein L₁ and L₂ may be the same or different, and wherein Z represents a divalent linker group of up to 10 atoms linking L₁ and L₂ and Z comprises at least 2 heteroatoms. In some embodiments Z comprises a 3-10 membered carboaromatic, a 3-10 membered carbocycle, a 3-10 membered heterocycle, a 3-10 membered heteroaromatic, an imide, an amidine, a guanidine, a 1,2,3-triazole, a sulfoxide, a sulfone, a thioester, a thioamide, a thiourea, an amide, an ester, a carbamate, a carbonate ester or a urea. In some embodiments Z represents a carbonyl-containing group. In some embodiments Z comprises an amide or an ester. Preferably Z is an amide. In some embodiments L₁ comprises —(OCH₂CH₂)ₚ—, wherein p is a number in the range 1 to 10, e.g. 2, 3, 4, or 5. In some embodiments L₂ comprises —(OCH₂CH₂)_{q}—, wherein q is a number in the range 1 to 10, e.g. 5, 6, 7, 8, 9 or 10.

In some embodiments MTX-L- is of the formula:

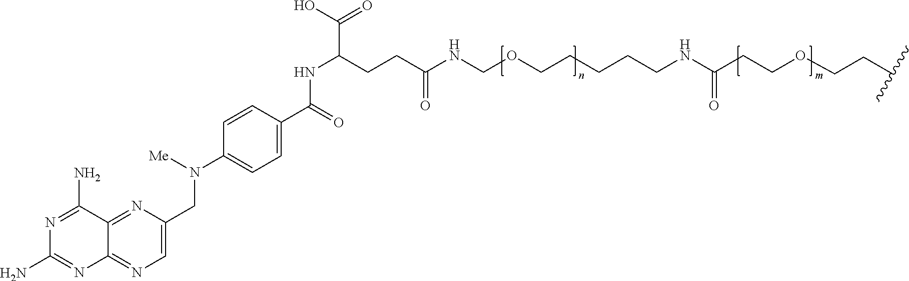

wherein n and m are independently 1, 2, 3, 4, 5, 6, 7, 8, 9 or 10.

In some embodiments MTX-L- is of the formula:

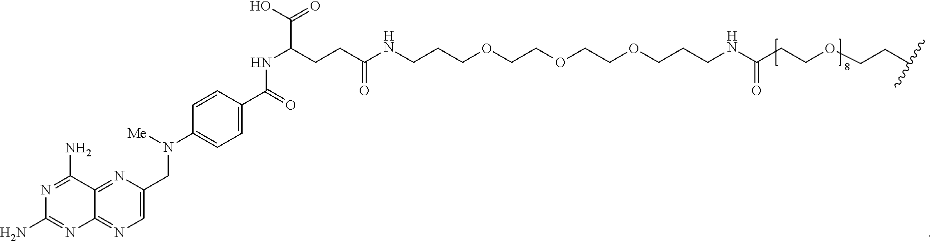

In some embodiments MTX-L- is of the formula:

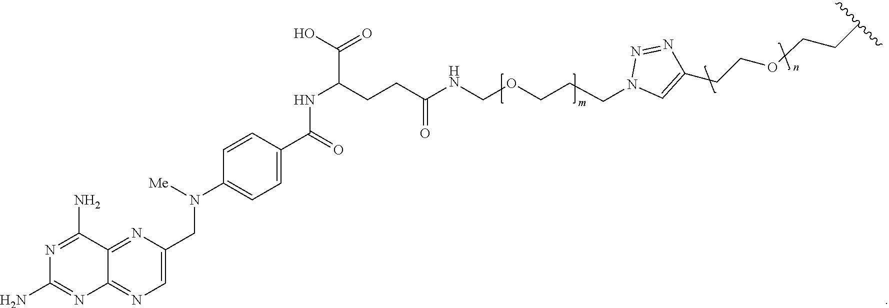

In some embodiments MTX-L- is of the formula:

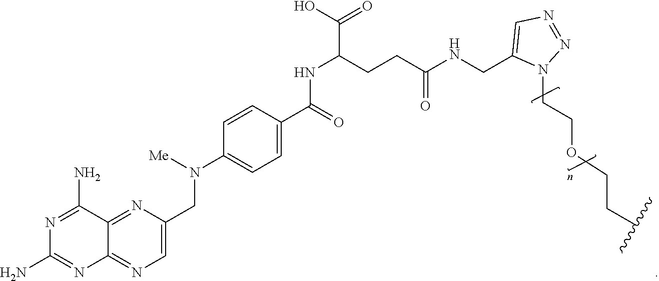

In some embodiments MTX-L- is of the formula:

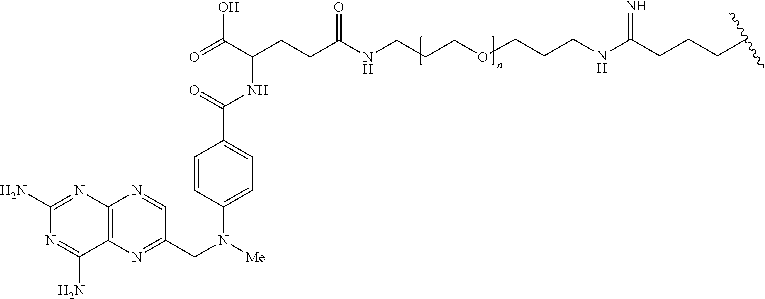

In particular embodiments MTX-L comprises a terminal thiol group;

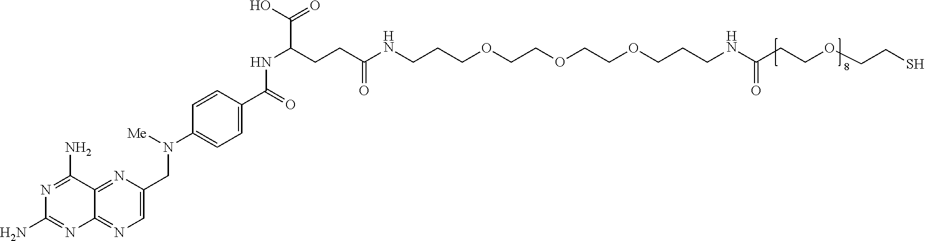

which terminal thiol group is bound to, e.g., a gold atom present at the surface of said core, as depicted below:

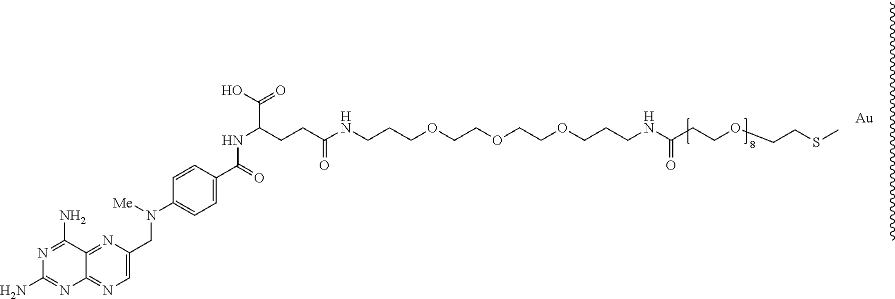

Other such embodiments of MTX-L- include:

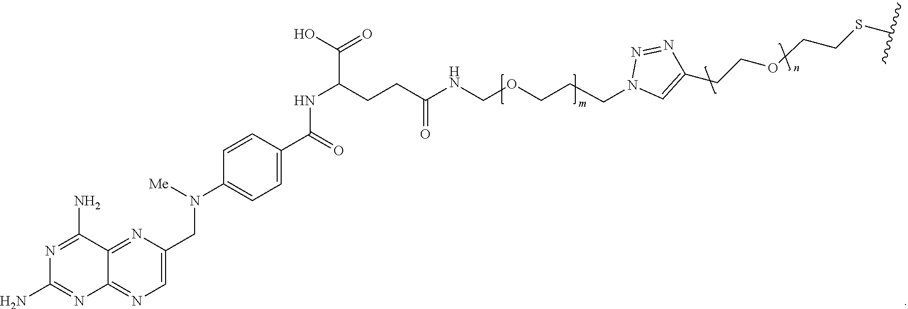

wherein n and m are independently 1, 2, 3, 4, 5, 6, 7, 8, 9 or 10;

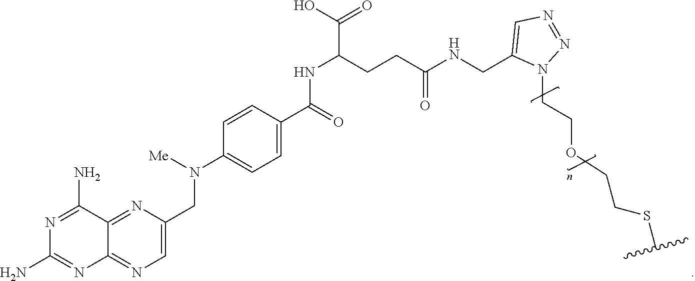

wherein n is an integer of between 1 and 15; and

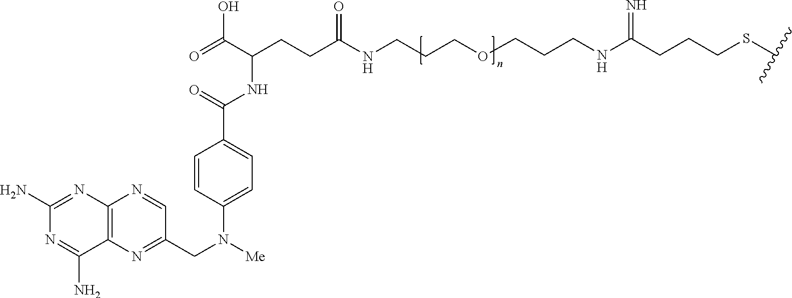

wherein n is an integer of between 1 and 15.

In certain embodiments MTX-L- is of the formula:

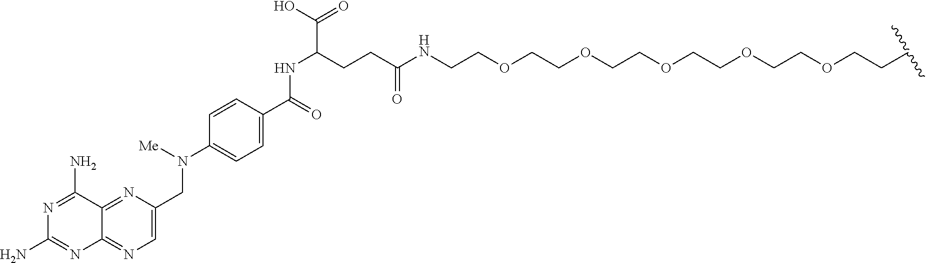 [SMILES: *CCOCCOCCOCCOCCOCCNC(=O)CCC(NC(=O)c1ccc(N(C)Cc2cnc3nc(N)nc(N)c3n2)cc1)C(=O)O]

In particular embodiments MTX-L comprises a terminal thiol group

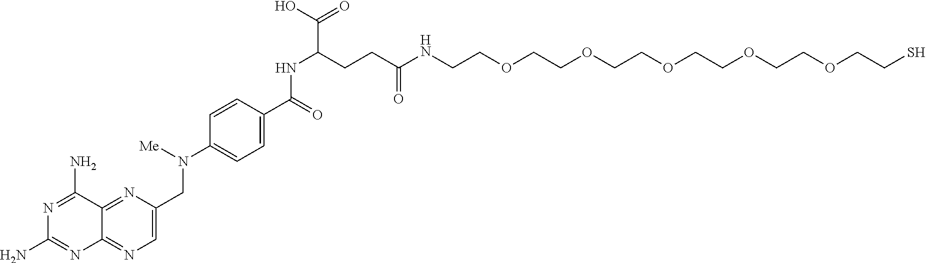 [SMILES: CN(Cc1cnc2nc(N)nc(N)c2n1)c1ccc(C(=O)NC(CCC(=O)NCCOCCOCCOCCOCCOCCS)C(=O)O)cc1]

which terminal thiol group is bound to, e.g., a gold atom present at the surface of said core, as depicted below:

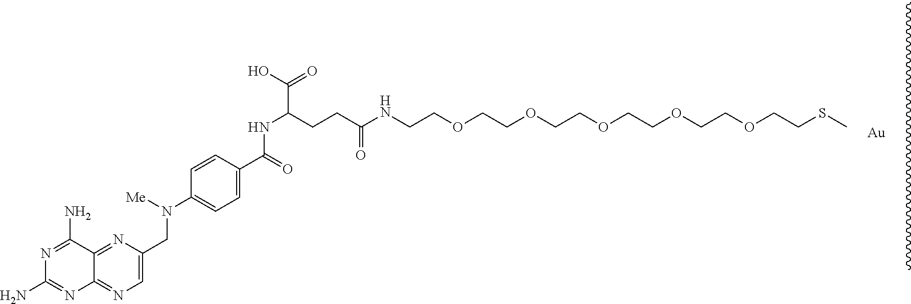

In some embodiments in accordance with any aspect of the present invention L may be bound to the core via a terminal sulphur atom.

In some embodiments, the nanoparticle may be of the formula: [dilution ligand]ₛ[MTX-L-S]ₜ@Au, wherein s and t are independently numbers >1. In some cases s may be >20. In some cases t may be >3, e.g., >5 or even >10. As used herein the formula of the general structure [ligand 1]ᵤ[ligand 2]_{c}@Au defines a gold nanoparticle having a number u of ligand 1 moieties and a number c of ligand 2 moieties covalently attached to its surface.

Typically, the nanoparticle will have unreacted linker ligands that have not had a methotrexate molecule coupled to them. Accordingly, in some embodiments the nanoparticle may be of the formula: [dilution ligand]ₛ[MTX-L-S]ₜ[COOH-L-S]ᵤ@Au or [dilution ligand]ₛ[MTX-L-S]ₜ[NH₂-L-S]ᵤ@Au, wherein s, t and u are independently numbers >1. In some cases s may be >20, e.g., >30. In some cases t may be >3, e.g., >5 or even >10. In some cases u may be >10, e.g., >20.

In some embodiments in accordance with any aspect of the present invention said dilution ligand may comprise a carbohydrate which is a monosaccharide or a disaccharide. In particular, the dilution ligand comprises galactose, glucose, mannose, fucose, maltose, lactose, galactosamine and/or N-acetylglucosamine.

In some embodiments the carbohydrate-containing dilution ligand may be covalently linked to the core via a C2-C15 (e.g. C2-C5) alkyl chain having a terminal thiol group. In particular embodiments the dilution ligand may comprise 2′-thioethyl-α-D-galactopyranoside or 2-thioethyl-β-D-glucopyranoside.

In some embodiments the core comprises a metal selected from the group consisting of: Au, Ag, Cu, Pt, Pd, Fe, Co, Gd, Zn or any combination thereof. In particular, the core may comprise gold.

In some embodiments the nanoparticle may be of the formula: [α-galactose-C2-S]ₛ[MTX-L-S]ₜ@Au, wherein s and t are independently numbers >1. In some cases s may be >20. In some cases t may be >3, e.g., >5 or even >10.

In some embodiments the nanoparticle may be of the formula: [α-galactose-C2-S]ₛ[MTX-L-S]ₜ[COOH-L-S]ᵤ@Au or [α-galactose-C2-S]ₛ[MTX-L-S]ₜ[NH₂-L-S]ᵤ@Au, wherein s, t and u are independently numbers >1. In some cases s may be >20, e.g., >30. In some cases t may be >3, e.g., >5 or even >10. In some cases u may be >10, e.g., >20.

In some embodiments the diameter of the core is in the range 1 nm to 5 nm, such as between 2 and 4 nm. The diameter of the core may be determined, for example, using electron microscopy or dynamic light scattering (DLS).

In some embodiments the diameter of the nanoparticle including its ligands is in the range 3 nm to 50 nm, such as 5 to 20 nm.

In some embodiments the total number of ligands per core is in the range 20 to 200.

In some embodiments the number of ligands of said formula MTX-L- per core is at least 3, such as at least 5, at least 10, at least 12 or at least 15. It may be in the range of 5-10, 10-15 or 15-20 per core.

In some embodiments the nanoparticle of the present invention has the MTX-L and dilution ligands as depicted in the following structure:

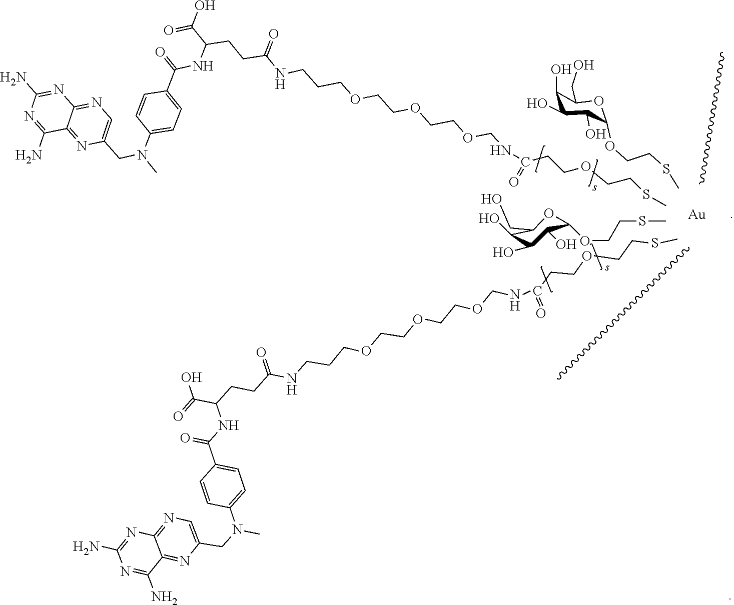

The nanoparticle size, ligand size, number of ratio of ligands is not depicted to scale. Other ligands not shown may be present. In some cases the total number of ligands per core is at least 5, and the total number of methotrexate-containing ligands per core is at least 5. Preferably, the total number of ligands per core is at least 10, 15 or 20. Preferably, the total number of methotrexate-containing ligands per core is at least 5, 10 or 15.

In some embodiments the nanoparticle of the present invention has the MTX-L and dilution ligands as depicted in the following structure:

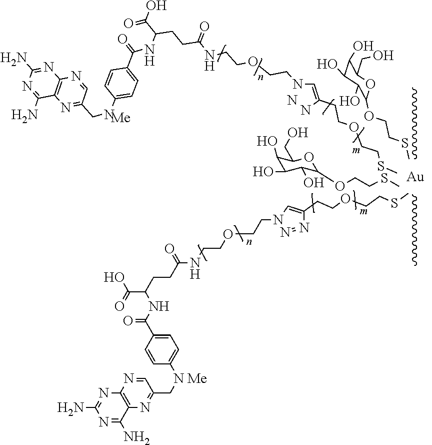

wherein n and m are independently 1, 2, 3, 4, 5, 6, 7, 8, 9 or 10, the total number of ligands per core is at least 5, and the total number of methotrexate-containing ligands per core is at least 3. Preferably, the total number of ligands per core is at least 10, 15 or 20. Preferably, the total number of methotrexate-containing ligands per core is at least 5, 10 or 15.

In some embodiments the nanoparticle of the present invention has the MTX-L and dilution ligands as depicted in the following structure:

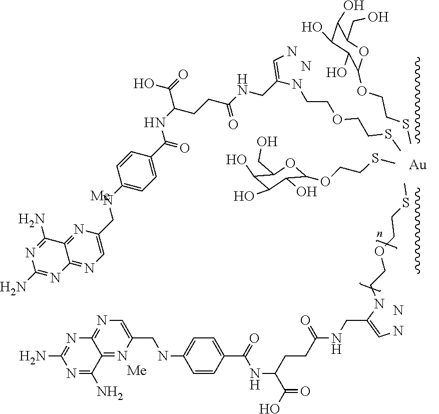

wherein n is an integer of between 1 and 15, the total number of ligands per core is at least 5, and the total number of methotrexate-containing ligands per core is at least 3. Preferably, the total number of ligands per core is at least 10, 15 or 20. Preferably, the total number of methotrexate-containing ligands per core is at least 5, 10 or 15.

In some embodiments the nanoparticle of the present invention has the MTX-L and dilution ligands as depicted in the following structure:

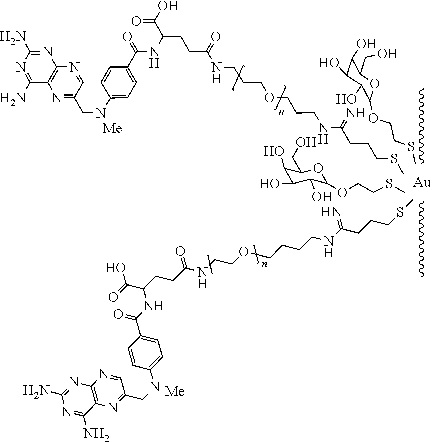

wherein n is an integer of between 1 and 15, the total number of ligands per core is at least 5, and the total number of methotrexate-containing ligands per core is at least 3. Preferably, the total number of ligands per core is at least 10, 15 or 20. Preferably, the total number of methotrexate-containing ligands per core is at least 5, 10 or 15.

In addition to the methods presented herein, co-pending application PCT/EP2019/085203, filed 13 Dec. 2019, provides further methods by which the claimed nanoparticles and their intermediates may be synthesised, and is incorporated herein by reference.

In a second aspect the present invention provides a pharmaceutical composition comprising a plurality of nanoparticles of the first aspect of the invention and at least one pharmaceutically acceptable carrier or diluent.

In some embodiments the pharmaceutical composition is in the form of a gel. The gel may be a hydrogel. Hydrogels suitable for topical administration (e.g. dermal delivery) are discussed in, for example, Li and Mooney, Nature Reviews Materials, 2016, Vol. 1, Article number: 16071 and Rehman and Zulfakar, Drug Dev Ind Pharm., 2014, Vol. 40(4), pp. 433-440, both of which are incorporated herein by reference.

In some embodiments the gel is selected from the group consisting of: Carbopol® 980, Carbopol® 974 and Carbopol® ETD 2020.

In some embodiments the concentration of methotrexate in said gel is in the range 0.5 mg/mL to 10 mg/mL, optionally about 2 mg/mL. The concentration of methotrexate may be determined by, for example, HPLC as described in Example 2 herein. As used herein, the concentration of methotrexate may be the concentration of methotrexate or a derivative thereof (such as MTX-(EG)ₙ-NH₂) that is covalently bound to the nanoparticle. It is specifically contemplated that the concentrations ranges referred to above exclude free methotrexate in the gel.

In some embodiments the nanoparticle core is of gold and the concentration of gold in said gel is in the range 1 mg/mL to 20 mg/mL, optionally about 4 mg/mL.

In some embodiments the composition is for topical (e.g. dermal) administration.

In some embodiments the composition is for systemic administration (e.g. subcutaneous injection).

In a third aspect the present invention provides a nanoparticle of the first aspect of the invention or a pharmaceutical composition of the second aspect of the invention for use in medicine.

In a fourth aspect the present invention provides a nanoparticle of the first aspect of the invention or a pharmaceutical composition of the second aspect of the invention for use in the treatment of an inflammatory or autoimmune disorder in a mammalian subject.

In some embodiments the inflammatory or autoimmune disorder may be selected from the group consisting of: psoriasis, psoriatic arthritis, scleroderma, rheumatoid arthritis, juvenile dermatomyositis, lupus, sarcoidosis, Crohn's disease, eczema and vasculitis.

In some embodiments the inflammatory or autoimmune disorder is a skin disorder. In particular, the disorder may be psoriasis (e.g. psoriasis vulgaris or pustular, inverse, napkin, nail, guttate, oral, or seborrheic-like psoriasis). In some embodiments the disorder may be selected from: Pityriasis rubra pilaris, cutaneous lichen, rosacea, alopecia areata, cutaneous lymphoma, an eczematous skin disorder (such as atopic dermatitis, cutaneous drug reaction, prurigo nodularis, or cutaneous mastocytosis), an autoimmune bullous skin disorder (such as pemphigus/pemphigoid, dermatitis herpetiformis, epidermolysis bullosa), cutaneous lupus, cutaneous vasculitis, Behcet's disease, sclerodermiform skin disease, a neutrophil mediated skin disease (such as pyoderma gangrenosum, sweet syndrome, hidradenitis suppurativa, SAPHO syndrome), a granulomatous skin disease (such as granuloma annulare, erythema annulare, erythema nodosum, sarcoidosis or necrobiosis lipoidica).

In some embodiments the nanoparticle or composition may be administered concurrently, sequentially or separately with a second anti-inflammatory agent. In particular, the second anti-inflammatory agent may comprise ciclosporin, hydroxycarbamide, dimethyl fumarate, a retinoid or biologic anti-inflammatory agent (e.g. an anti-TNFα antibody, an anti-TNFα decoy receptor, an anti-IL-17 antibody or an anti-IL-23 antibody).

In a fifth aspect the present invention provides a method of treating an inflammatory or autoimmune disorder in a mammalian subject, comprising administering a nanoparticle of the first aspect of the invention or a pharmaceutical composition of the second aspect of the invention to the subject in need of therapy.

In some embodiments the inflammatory or autoimmune disorder may be selected from the group consisting of: psoriasis, psoriatic arthritis, scleroderma, rheumatoid arthritis, juvenile dermatomyositis, lupus, sarcoidosis, Crohn's disease, eczema and vasculitis.

In some embodiments the inflammatory or autoimmune disorder is a skin disorder. In particular, the disorder may be psoriasis (e.g. psoriasis vulgaris or pustular, inverse, napkin, nail, guttate, oral, or seborrheic-like psoriasis). In some embodiments the disorder may be selected from: Pityriasis rubra pilaris, cutaneous lichen, rosacea, alopecia areata, cutaneous lymphoma, an eczematous skin disorder (such as atopic dermatitis, cutaneous drug reaction, prurigo nodularis, or cutaneous mastocytosis), an autoimmune bullous skin disorder (such as pemphigus/pemphigoid, dermatitis herpetiformis, epidermolysis bullosa), cutaneous lupus, cutaneous vasculitis, Behcet's disease, sclerodermiform skin disease, a neutrophil mediated skin disease (such as pyoderma gangrenosum, sweet syndrome, hidradenitis suppurativa, SAPHO syndrome), a granulomatous skin disease (such as granuloma annulare, erythema annulare, erythema nodosum, sarcoidosis or necrobiosis lipoidica).

In a sixth aspect the present invention provides use of a nanoparticle of the first aspect of the invention or a pharmaceutical composition of the second aspect of the invention in the preparation of a medicament for use in a method of the fifth aspect of the invention.

In a seventh aspect the present invention provides an article of manufacture comprising:
a nanoparticle of the first aspect of the invention or a pharmaceutical composition of the second aspect of the invention;
a container for housing the nanoparticle or pharmaceutical composition; and
an insert or label.

In some embodiments the insert and/or label provides instructions, dosage and/or administration information relating to the use of the nanoparticle or pharmaceutical composition in a method of treatment of the fifth aspect of the invention.

In accordance with any aspect of the present invention, the subject may be a human, a companion animal (e.g. a dog or cat), a laboratory animal (e.g. a mouse, rat, rabbit, pig or non-human primate), a domestic or farm animal (e.g. a pig, cow, horse or sheep).

Preferably, the subject is a human who has been diagnosed as having psoriasis (e.g. psoriasis vulgaris or pustular, inverse, napkin, guttate, oral, or seborrheic-like psoriasis). In some embodiments the subject may have or may have previously had psoriasis, but may currently be in remission and the nanoparticle or composition for use, the method or the use of the invention may be for prophylactic treatment of psoriasis or to delay or prevent recurrence of psoriasis.

The nanoparticle or composition of the invention may be for application directly to an affected site (e.g. topical application to a psoriatic lesion) and/or for application to a so-far unaffected site or a site in remission (e.g. non-inflamed skin).

Embodiments of the present invention will now be described by way of example and not limitation with reference to the accompanying figures. However various further aspects and embodiments of the present invention will be apparent to those skilled in the art in view of the present disclosure.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1 depicts the general chemical structure of a gold core nanoparticle having a corona comprising alpha-galactose-C2-SH ligands and MTX-PEG₃NHC(O)PEG₈-SH ligands, also described herein as MTX-PEG₃-NH₂-loaded GNPs.

FIG. 2: Systemic MTX in the IMQ-induced mouse model.

(a) Experimental Scheme of three-day IMQ treatment with seven-day systemic therapy. (b) (upper panel) change in ear thickness between control (PBS; crosses), IMQ-treated (squares), and systemic MTX therapy receiving (1 mg/kg (upward triangles), 2 mg/kg (downward triangles), 5 mg/kg (diamonds)) animals. (Lower panels) statistical analysis of ear thickness differences between groups between days 4-7. (c) Weight changes of mice were calculated as % weight change from pre-treatment weight and recorded daily (upper panel), and plotted for day 7 (lower panel). ns=non-significant, *=p<0.05, **=p<0.01, ***=p<0.001 ****=p<0.0001.

FIG. 3: Systemic MTX vs. MTX-GNP in the IMQ-induced mouse model.

(a) (upper panel), change in ear thickness between control (PBS, crosses), IMQ-treated (squares), and systemic therapy receiving in addition to IMQ, 2 mg/kg MTX (upward triangles), GNPs containing 5.5 mg/kg Au (downward triangles), and MTX-GNP containing 2 mg/kg MTX and 5.5 mg/kg Au (diamonds). (Lower panels) statistical analysis of ear thickness differences between groups between days 4-7. (b) Weight changes of mice were calculated as % weight change from pre-treatment weight and recorded daily (upper panel; control (PBS, crosses), IMQ-treated (squares), and systemic therapy receiving in addition to IMQ, 2 mg/kg MTX (upward triangles), GNPs containing 5.5 mg/kg Au (downward triangles), and MTX-GNP containing 2 mg/kg MTX and 5.5 mg/kg Au (diamonds)), and plotted for day 7 (lower panel; bars left to right: untreated, IMQ, MTX systemic, GNP systemic, MTX-GNP systemic). Data is pooled from 2-3 independent experiments, 2 to 5 mice per condition and represented as mean±standard deviation. (c) Cell counts of CD45⁺ cell in ears of (left to right): untreated, IMQ, MTX systemic, GNP systemic and MTX-GNP systemic treated mice are plotted and statistical comparisons shown. ns=non-significant, *=p<0.05, **=p<0.01, ***=p<0.001 ****=p<0.0001.

FIG. 4: Topical MTX vs. MTX-GNP in the IMQ-induced mouse model.

(a) Experimental Scheme of three-day IMQ treatment with seven-day topical therapy. (b) Representative hematoxylin eosin staining of ear skin of mice at day 8 for (left to right): untreated, IMQ, IMQ+MTX, IMQ+GNP and IMQ+MTX-GNP. Scale bar=200 μm. (c) (Upper panel) change in ear thickness between control (PBS, crosses), IMQ-treated (squares), and topical therapy receiving Carbopol 980 gel carrier (circles), Carbopol 980 gel containing 12.5 mg/kg MTX (upward triangles), Carbopol 980 gel containing GNPs 37.5 mg/kg Au (downward triangles), and Carbopol 980 gel containing MTX-GNPs 12.5 mg/kg MTX and 37.5 mg/kg Au (diamonds) animals. (Lower panels) statistical analysis of ear thickness differences between groups between days 4-7. (d) Weight changes of mice were calculated as % weight change from pre-treatment weight and recorded daily (upper panel; symbols as for (c)) and plotted for day 7 (lower panel; left to right: untreated, IMQ, carrier, topical MTX 12.5 mg/kg MTX, topical GNP 37.5 mg/kg GNP and topical MTX-GNP 12.5 mg/kg MTX). Data is pooled from 3 independent experiments, 2 to 5 mice per condition and represented as mean±standard deviation. (e) Flow cytometry analysis of immune infiltration in ear skin upon different topical therapies. Representative FACS plots of CD45+ cell populations into ears upon different topical therapies (left to right: untreated, IMQ, IMQ+topical MTX 12.5 mg/kg MTX, IMQ+topical GNP 37.5 mg/kg GNP and IMQ+topical MTX-GNP 12.5 mg/kg MTX. (f) Cell counts of CD45⁺ cell in ears of (left to right): untreated, IMQ, MTX topical, GNP topical and MTX-GNP topical treated mice are plotted and statistical comparisons shown. ns=non-significant, *=p<0.05, **=p<0.01, ***=p<0.001 ****=p<0.0001.

FIG. 5: Flow cytometry analysis of immune infiltration in ear skin upon different topical therapies.

(a) Representative FACS plots of CD3+ CD11b+ cell populations into ears upon different topical therapies (left to right): untreated, IMQ, IMQ+MTX topical, IMQ+GNP topical and IMQ+MTX-GNP topical. (b) Quantification of CD3⁺ cell in ears. Cell counts plotted for (left to right): untreated, IMQ, IMQ+MTX topical, IMQ+GNP topical and IMQ+MTX-GNP topical. Statistical comparisons shown. (c) (left panel) Quantification of CD11b⁺ cell in ears. Cell counts plotted for (left to right): untreated, IMQ, IMQ+MTX topical, IMQ+GNP topical and IMQ+MTX-GNP topical. Statistical comparisons shown. (Right panel) Ratio of CD3⁺:CD11b⁺ cells plotted for (left to right): untreated, IMQ, IMQ+MTX topical, IMQ+GNP topical and IMQ+MTX-GNP topical. Statistical comparisons shown. (d) Comparison of cell counts of α and γδ T cell composition of CD3⁺ cells in ears. (e) Comparison of cell counts of CD4⁺ and CD8⁺ T cell composition of αβ CD3⁺ cells in ears. (f) (Upper panels) Representative FACS plots of Ly6G⁺ CD11b⁺ cell populations into ears upon different topical therapies (left to right: untreated, IMQ, IMQ+MTX topical, IMQ+GNP topical and IMQ+MTX-GNP topical. (Lower panel) Comparison of Ly6G⁺ vs. Ly6G⁻ cells for the indicated treatments. ns=non-significant, *=p<0.05, **=p<0.01, ***=p<0.001 ****=p<0.0001.

FIG. 6: Flow cytometry analysis of immune cells in spleen upon different systemic and topical therapies.

(a) Cell counts of CD45⁺ cells in spleen for systemic (left) and topical (right) treatment with the indicated treatments. (b) Cell counts of CD3⁺ cells in spleen following the indicated topical treatments. (c) Cell counts of CD11b⁺ cells in spleen of the indicated topical treatments. (d) Comparison of cell counts of a and γδ T cell composition of CD3⁺ cells in spleen following the indicated topical treatments. (e) Comparison of cell counts of CD4⁺ and CD8⁺ T cell composition of αβ CD3⁺ cells in spleen following the indicated topical treatments. (f) Comparison of Ly6G⁺ vs. Ly6G⁻ cells in spleen following the indicated treatments. ns=non-significant, *=p<0.05, **=p<0.01, ***=p<0.001 ****=p<0.0001.

FIG. 7: Acanthosis (skin thickening) plotted in μm for ears of (left to right): untreated, IMQ, IMQ+topical MTX gel, IMQ+topical GNP gel and IMQ+MTX-GNP gel. Statistical comparisons shown. ns=non-significant, *=p<0.05, **=p<0.01, ***=p<0.001 ****=p<0.0001.

FIG. 8: Acanthosis (skin thickening) plotted in μm for ears of AGR129 xenotransplantation human skin mouse model (left to right): vaseline, Daivobet, and MTX-GNP gel. Statistical comparisons shown. ns=non-significant, *=p<0.05, **=p<0.01, ***=p<0.001 ****=p<0.0001.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

In describing the present invention, the following terms will be employed, and are intended to be defined as indicated below.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

Nanoparticles

As used herein, “nanoparticle” refers to a particle having a nanomeric scale, and is not intended to convey any specific shape limitation. In particular, “nanoparticle” encompasses nanospheres, nanotubes, nanoboxes, nanoclusters, nanorods and the like. In certain embodiments the nanoparticles and/or nanoparticle cores contemplated herein have a generally polyhedral or spherical geometry. References to “diameter” of a nanoparticle or a nanoparticle core a generally taken to mean the longest dimension of the nanoparticle or nanoparticle core, respectively. For nanoparticles having a substantially polyhedral or spherical geometry, the shortest dimension across the particle will typically be within 50% of the longest dimension across the particle and may be, e.g., within 25% or 10%.

Nanoparticles comprising a plurality of carbohydrate-containing ligands have been described in, for example, WO 2002/032404, WO 2004/108165, WO 2005/116226, WO 2006/037979, WO 2007/015105, WO 2007/122388, WO 2005/091704 (the entire contents of each of which is expressly incorporated herein by reference) and such nanoparticles may find use in accordance with the present invention.

As used herein, “corona” refers to a layer or coating, which may partially or completely cover the exposed surface of the nanoparticle core. The corona includes a plurality of ligands covalently attached to the core of the nanoparticle. Thus, the corona may be considered to be an organic layer that surrounds or partially surrounds the metallic core. In certain embodiments the corona provides and/or participates in passivating the core of the nanoparticle. Thus, in certain cases the corona may include a sufficiently complete coating layer substantially to stabilise the core. In certain cases the corona facilitates solubility, such as water solubility, of the nanoparticles of the present invention.

Nanoparticles are small particles, e.g. clusters of metal or semiconductor atoms, that can be used as a substrate for immobilising ligands.

Preferably, the nanoparticles have cores having mean diameters between 0.5 and 50 nm, more preferably between 0.5 and 10 nm, more preferably between 0.5 and 5 nm, more preferably between 0.5 and 3 nm and still more preferably between 0.5 and 2.5 nm. When the ligands are considered in addition to the cores, preferably the overall mean diameter of the particles is between 2.0 and 50 nm, more preferably between 3 and 10 nm and most preferably between 4 and 5 nm. The mean diameter can be measured using techniques well known in the art such as transmission electron microscopy.

The core material can be a metal or semiconductor and may be formed of more than one type of atom. Preferably, the core material is a metal selected from Au, Fe or Cu. Nanoparticle cores may also be formed from alloys including Au/Fe, Au/Cu, Au/Gd, Au/Fe/Cu, Au/Fe/Gd and Au/Fe/Cu/Gd, and may be used in the present invention. Preferred core materials are Au and Fe, with the most preferred material being Au. The cores of the nanoparticles preferably comprise between about 100 and 500 atoms or 100 to 2,000 atoms (e.g. gold atoms) to provide core diameters in the nanometre range. Other particularly useful core materials are doped with one or more atoms that are NMR active, allowing the nanoparticles to be detected using NMR, both in vitro and in vivo. Examples of NMR active atoms include Mn⁺², Gd⁺³, Eu⁺², Cu⁺², V⁺², Co⁺², Ni⁺², Fe⁺², Fe⁺³ and lanthanides⁺³, or the quantum dots.

Nanoparticle cores comprising semiconductor compounds can be detected as nanometre scale semiconductor crystals, and are capable of acting as quantum dots, that is they can absorb light thereby exciting electrons in the materials to higher energy levels, subsequently releasing photons of light at frequencies characteristic of the material. An example of a semiconductor core material is cadmium selenide, cadmium sulphide, cadmium telluride. Also included are the zinc compounds such as zinc sulphide.

In some embodiments, the nanoparticle or its ligand comprises a detectable label. The label may be an element of the core of the nanoparticle or the ligand. The label may be detectable because of an intrinsic property of that element of the nanoparticle or by being linked, conjugated or associated with a further moiety that is detectable.

Methotrexate

Methotrexate (MTX), formerly known as amethopterin, is a chemotherapy agent and immune system suppressant. It has found use in the treatment of various cancers, autoimmune diseases, ectopic pregnancy, and for medical abortions.

MTX has the CAS number 59-05-2 and has the structure depicted below:

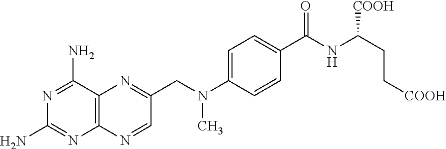 [SMILES: CN(Cc1cnc2nc(N)nc(N)c2n1)c1ccc(C(=O)N[C@@H](CCC(=O)O)C(=O)O)cc1]

As used herein “methotrexate” or “MTX” refers to not only the compound of the of the above chemical formula, but also derivatives of MTX in which one or more functional groups have been modified for attachment to the nanoparticle via the linker L. In particular, MTX may be bonded to linker L via, e.g., an amide formed at a carboxylic acid group in the above structure.

Ethylene Glycol

As used herein, an ethylene glycol-containing linker or chain means that one or more ethylene glycol subunits is present. This may be depicted or represented in a variety of ways, such as —(OCH₂CH₂)ₘ— or (EG)ₘ or (PEG)ₘ or PEGₘ or PEGm, where m is a number. Unless context dictates otherwise, these terms are used interchangeably herein.

Thus, the term “PEG” may be employed herein to mean shorter, e.g., oligomer length chains of ethylene glycol units, such as PEG3 or PEG8, which have the same meaning as (EG)₃ and (EG)₈, respectively.

Gel

A gel is a non-fluid colloidal network or polymer network that is expanded throughout its volume by a fluid. In the present context, the gel may be a pharmaceutically acceptable gel, e.g., a hydrogel. A particularly suitable class of hydrogels are hydrogels formed of the Carbopol® family of crosslinked polyacrylic acid polymers available from Lubrizol Corporation and described at https://www.lubrizol.com/Life-Sciences/Products/Carbopol-Polymer-Products.

Administration and Treatment

The nanoparticles and compositions of the invention may be administered to patients by any number of different routes, including enteral or parenteral routes. Parenteral administration includes administration by the following routes: intravenous, cutaneous or subcutaneous, nasal, intramuscular, intraocular, transepithelial, intraperitoneal and topical (including dermal, ocular, rectal, nasal, inhalation and aerosol), and rectal systemic routes. A preferred route of administration is dermal administration by topical application to the skin.

The nanoparticles of the invention may be formulated as pharmaceutical compositions that may be in the forms of solid or liquid compositions. Such compositions will generally comprise a carrier of some sort, for example a solid carrier or a liquid carrier such as water, petroleum, animal or vegetable oils, mineral oil or synthetic oil. Physiological saline solution, or glycols such as ethylene glycol, propylene glycol or polyethylene glycol may be included. Such compositions and preparations generally contain at least 0.1 wt % of the compound.

For intravenous, cutaneous or subcutaneous injection, or injection at the site of affliction, the active ingredient will be in the form of a parenterally acceptable aqueous solution or liquid which is pyrogen-free and has suitable pH, tonicity and stability. Those of relevant skill in the art are well able to prepare suitable solutions using, for example, solutions of the compounds or a derivative thereof, e.g. in physiological saline, a dispersion prepared with glycerol, liquid polyethylene glycol or oils.

In addition to one or more of the compounds, optionally in combination with another active ingredient, the compositions can comprise one or more of a pharmaceutically acceptable excipient, carrier, buffer, stabiliser, isotonicising agent, preservative or anti-oxidant or other materials well known to those skilled in the art. Such materials should be non-toxic and should not interfere with the efficacy of the active ingredient. The precise nature of the carrier or other material may depend on the route of administration, e.g., topical application or intravenous injection.

Preferably, the pharmaceutically compositions are given to an individual in a prophylactically effective amount or a therapeutically effective amount (as the case may be, although prophylaxis may be considered therapy), this being sufficient to show benefit to the individual. Typically, this will be to cause a therapeutically useful activity providing benefit to the individual. The actual amount of the compounds administered, and rate and time-course of administration, will depend on the nature and severity of the condition being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Handbook of Pharmaceutical Additives, 2nd Edition (eds. M. Ash and I. Ash), 2001 (Synapse Information Resources, Inc., Endicott, New York, USA); Remington's Pharmaceutical Sciences, 20th Edition, 2000, pub. Lippincott, Williams & Wilkins; and Handbook of Pharmaceutical Excipients, 2nd edition, 1994. By way of example, the compositions are preferably administered to patients in dosages of between about 0.01 and 100 mg of active compound per kg of body weight, and more preferably between about 0.5 and 10 mg/kg of body weight. In the context of treatment of a skin disorder, one benefit of topical administration of a composition of the present invention is that the resulting systemic concentration of methotrexate will be significantly lower than if methotrexate were administered systemically. This means that toxic and other unwanted side effects of methotrexate can be minimised or substantially avoided while nevertheless achieving clinically beneficial concentrations of methotrexate at the affected site(s) of the subject's skin.

The following is presented by way of example and is not to be construed as a limitation to the scope of the claims.

### EXAMPLES

### Example 1—Synthesis of Methotrexate-Coupled Gold Nanoparticles (MTX-GNPs)

### Preparation of Ligands and Synthesis of [α-Gal]₂₂[AL]₂₂@Au GNPs

Gold nanoparticles having a corona of alpha-galactose-C2 (α-Gal) and 1-amino-6-mercapto-hexaethylenglycol (SH—CH₂-(EG)₆-NH₂ also known as “amino linker” or “AL”) ligands were synthesised as described previously (see WO2011/154711, Examples 1 and 2, and WO2016/102613, Example 1, both of which documents are incorporated herein by reference).

### Preparation of 2-thio-ethyl-α-d-galactoside (α-galactose-C2SH “α-Gal”)

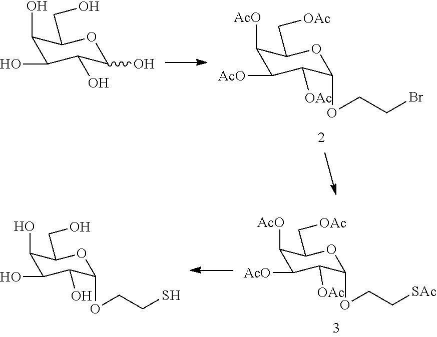

To a suspension of galactose (3 g, 16.65 mmol) in 2-bromoethanol (30 ml), acid resin Amberlite 120-H is added to reach pH 2. The reaction is stirred for 16 hours at 50-60° C. The reaction mixture is filtered and washed with MeOH. Triethylamine is added to reach pH 8. The crude of the reaction is concentrated and co evaporated 3 times with toluene. The reaction mixture is dissolved pyridine (75 mL) and Ac₂O (35 mL) and a catalytic amount of DMAP are added at 0° C. and stirred for 3 h at rt. The mixture is diluted with AcOEt and washed with 1.H₂O; 2.HCl (10%) 3. NaHCO₃ dis 4. H₂O. The organic layer is collected and dried over anhydrous Na₂SO₄. TLC (Hexane:AcOEt 3:1, 2 elutions) shows a major product (desired) and a lower Rf minority. The product is purified by flash chromatography using the mixture hexane:ethyl acetate 6:1 as eluent and the 2-bromoethyl-alpha-galactoside (2) is obtained.

The product of the previous reaction, 2 is dissolved in 27 ml of 2-butanone. To this solution, a catalytic amount of tetrabutylammonium iodide and 4 equivalents of potassium thioacetate are added. The resulting suspension is stirred for 2 hours at room temperature. Throughout this period the reaction is tested by TLC (hexane-AcOEt 2:1, 2 elutions) for the disappearance of the starting material. The mixture is diluted with 20 ml of AcOEt and washed with a saturated NaCl solution. The organic phase is dried, filtered and evaporated under vacuum. The product is purified in hexane/AcOEt 2:1→1:1 to obtain the acetylthio-alpha-galactoside 3.

The new product of the reaction, 3 is dissolved in a mixture dichloromethane-methanol 2:1. To this mixture a solution of 1N sodium methoxide (1 equivalent) is added and stirred for 1 hour at room temperature. Amberlite IR-120H resin is added to achieve pH 5-6. The resulting mixture is then filtered and concentrated to dryness to obtain the final product (α-galactose C2SH).

Preparation of Amino-Thiol Linker (AL)

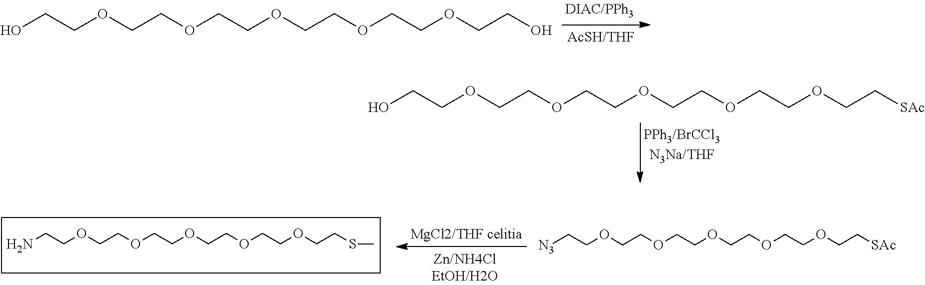

To a solution of PPh₃ (3 g, 11.4 mmol) in 20 ml dry THF, DIAC (2.3 g, 11.4 mmol) is added. The mixture is allowed to stir at 0° C. 15 min until the appearance of a white product. To this mixture a solution of hexaethyleneglycol (1.45 mL, 5.7 mmol) and HSAc (610 μl, 8.55 mmol) in dry THF (20 mL) is added dropwise (addition funnel). After 15 min the products begin to appear on TLC at Rf 0.2. The solution is concentrated in an evaporator. The crude of the reaction is dissolved in 50 ml of dichloromethane and washed with a solution of K₂CO₃ 10%. The organic phase is dried over anhydrous Na₂SO₄, filtered and concentrated under vacuum. Flash chromatography of the crude using AcOEt:Hexane 1:1, AcOEt and finally DCM:MeOH 4:1 as eluent gave the acetyl-thio-hexaethyleneglycol derivative.

The reaction product is dissolved in 5 ml of DMF and PPh₃ (2.25 g, 8.55 mmol), NaN₃ (0.741 g, 11.4 mmol) and BrCl₃C (0,845 ml, 8.55 mmol) are added and the solution subsequently stirred for 40 min at room temperature. The resulting product has a higher Rf than the starting product when performing TLC (DCM:MeOH 25:1). The reaction mixture is diluted with 100 ml of diethylether and washed three times with H₂O. The organic phase is dried over anhydrous Na₂SO₄, filtered and evaporated under vacuum. The product is purified by flash chromatography using the mixture of eluents DMC/MeOH 200:1 and DCM/MeOH 40:1 to obtain the azido-acetylthio-hexaethyleneglycol derivative.

To remove the triphenyl phosphine oxide, the reaction product is dissolved in 10 ml of THF and 0.5 g of MgCl₂ is added to this solution. The reaction is stirred for 2 h at 80° C. until a white precipitate appears and then is filtered through celite. The product is dissolved in a mixture of ethanol:H₂O 3:1 and added Zn dust (0.45 g, 6.84 mmol) and NH₄Cl (0.6 g, 11.4 mmol). The reaction was stirred at reflux for 1 h until the presence of starting material is no longer detectable by TLC (DCM/MeOH 25:1). The reaction is filtered through celite and the solvent is evaporated. The crude de reaction is diluted with AcOEt and extract with 5 ml H₂O. The aqueous phase is evaporated to dryness to obtain the amino-thiol-hexaethylenglycol product.

### Synthesis of [α-Gal]22[AL]22@Au GNPs

Alpha-galactose C2 derivative 3 and hexaethyleneglycol amine linker 6 were taken from Midatech Biogune stock. N-(3-Dimethylaminopropyl)-N′-ethylcarbodiimide hydrochloride (EDC-HCl), HAuCl₄, NaBH₄ were purchased from Sigma-Aldrich Chemical Company. Imidazole-4-acetic acid monohydrochloride was purchased from Alfa Aesar. Company High quality MeOH and Nanopure water (18.1 mΩ) were used for all experiments and solutions.

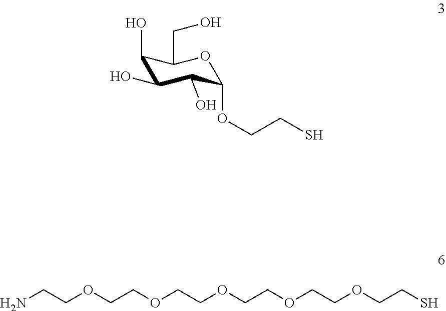

To a mix of amine-mercapto hexaethylenglycol linker 6 and alpha-galactose ligand 3 in a ratio 1:1 (0.58 mmol, 3 eq.) in MeOH (49 mL) was added an aqueous solution of gold salt (7.86 mL, 0.19 mmol, 0.025M). The reaction was stirred for 30 seconds and then, an aqueous solution of NaBH₄ (1N) was added in several portions (4.32 mL, 4.32 mmol). The reaction was shaken for 100 minutes at 900 rpm. After this time, the suspension was centrifuged 1 minute at 14000 rpm. The supernatant is removed and the precipitated was dissolved in 2 mL of water. Then, 2 mL of the suspension were introduced in two filters (Amicon, 10 KDa, 4 mL) and were centrifuged 5 minutes at 4500 g. The residue in the filter was washed twice more with water.

The final residue was dissolved in 80 mL of water.

### Functionalisation of [α-Gal]₂₂[AL]₂₂@Au GNPs with Methotrexate

Functionalisation of the [α-Gal]₂₂[AL]₂₂@Au nanoparticles prepared as described above with methotrexate was performed using 1-Ethyl-3-(3-dimethylaminopropyl)carbodiimide (EDC) and N-hydroxysuccinimide (NHS) in dimethyl sulfoxide (DMSO) at room temperature according to the following scheme:

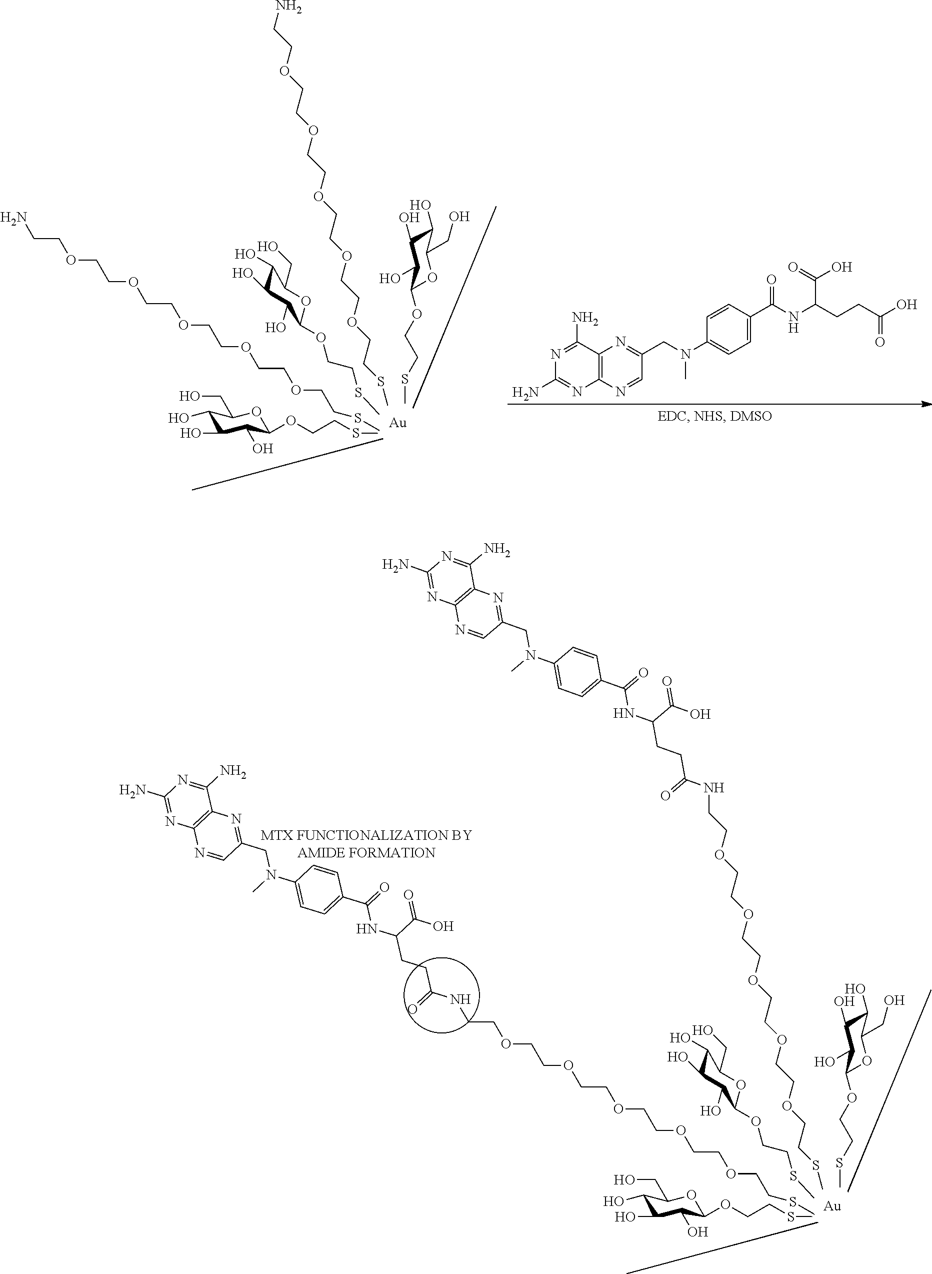

| Material | Supplier | Batch No. |
|---|---|---|
| Nanoparticle | Midatech Pharma | M199-082 |
| EDC | SIGMA-ALDRICH | S2BK8745V |
| NHS | ALDRICH | MKBP79891V |
| MTX | AVACHEM | ZW0701 |
| DMSO | SIGMA-ALDRICH | SHB61596V |

Procedure

The nanoparticles were concentrated by centrifugation and collected with DMSO (3.62 mL) to obtain about 8000 ppm of gold concentration.

Drug Activation

To a solution of MTX (0.1M) in DMSO, EDC (38.4 μL; 0.5M) was added and the mixture was stirred about five minutes. Then, NHS (19.2 μL; 1.0M) was added and the mixture was activated for thirty minutes at room temperature.

Drug Functionalization

[α-Gal]₂₂[AL]₂₂@Au GNPs (750 μL) were added to the previously activated solution and the coupling was incubated overnight at room temperature in darkness.

Purification

The nanoparticles were purified by centrifugation (4500 rpm, 10 min) using NaOH 0.1M as eluent. The content was collected in 500 μL H₂O (12.00 μg/μL) and was stored for further analysis.

Analysis

Gold content was assessed by inductively coupled plasma mass spectrometry (ICP-MS), size by dynamic light scattering (DLS) electrostatic charge by zeta potential, and structure by 1H NMR.

DLS size indicated a main peak at 5.15 nm. However, a secondary peak at 1.61 nm was also observed indicating two populations of nanoparticles. Differential centrifugation sedimentation (DCS) analysis confirmed the presence of two populations of nanoparticles, with sizes of 3.0 nm and 8.0 nm.

Zeta potential was found to be −51.1 mV (i.e. negatively charged).

The above procedure was repeated with different equivalents of MTX. In each case the final loading of MTX per nanoparticle was determined by 1H NMR analysis. MTX loadings from 2 equivalents/GNP up to ˜5 equivalents/GNP were obtained.

Conclusions

The above results demonstrate successful synthesis of [α-Gal]-[MTX-AL]@Au GNPs with size <10 nm and up to 5 equivalents of MTX per GNP. However, variability was observed between batches for GNP size and zeta potential. Methotrexate has two potential carboxylate binding sites that may lead to variability in binding capacity to the amine groups on positively charged GNPs (i.e. possible dual EDC activation of MTX may explain a heterogeneous product).

### Example 2—Synthesis of Modified Methotrexate-Coupled Gold Nanoparticles (MTX-GNPs)

The present inventors aimed to increase the MTX loading per GNP and to reduce variability due to the multiple carboxyl groups on MTX observed in Example 1.

To this end, a modified methotrexate having a (EG)₃NH₂ linker was synthesised as described in co-pending application GB1820470.1, filed 18 Dec. 2018 (see Example 2 thereof, which is expressly incorporated herein by reference).

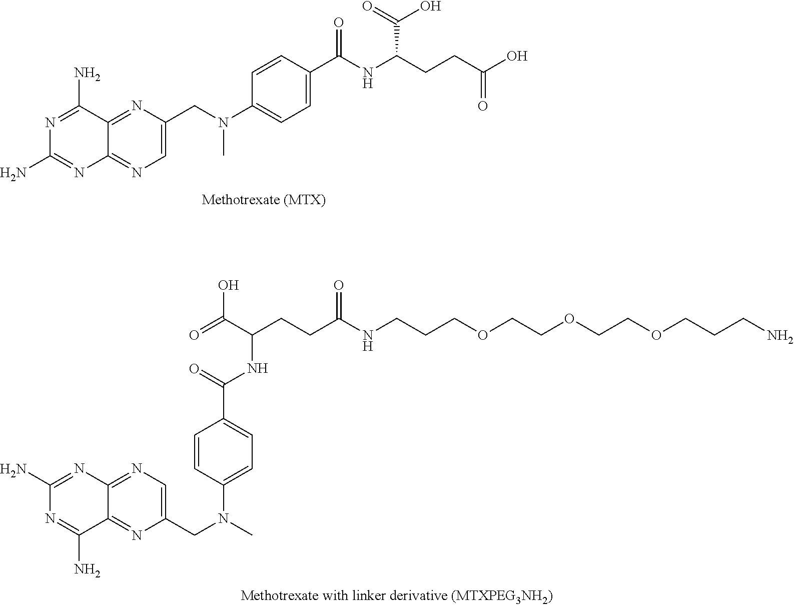 [SMILES: CN(Cc1cnc2nc(N)nc(N)c2n1)c1ccc(C(=O)N[C@@H](CCC(=O)O)C(=O)O)cc1 ; CN(Cc1cnc2nc(N)nc(N)c2n1)c1ccc(C(=O)NC(CCC(=O)NCCCOCCOCCOCCCN)C(=O)O)cc1]

The chemical name of the methotrexate derivative with linker is 4-[(3-{2-[2-(3-aminopropoxy)ethoxy]ethoxy}propyl)carbamoyl]-2-[(4-{[(2,4-diaminopteridin-6-yl)methyl](methyl)amino}phenyl)formamido]butanoic acid. The methotrexate derivative was prepared according to the following reaction scheme:

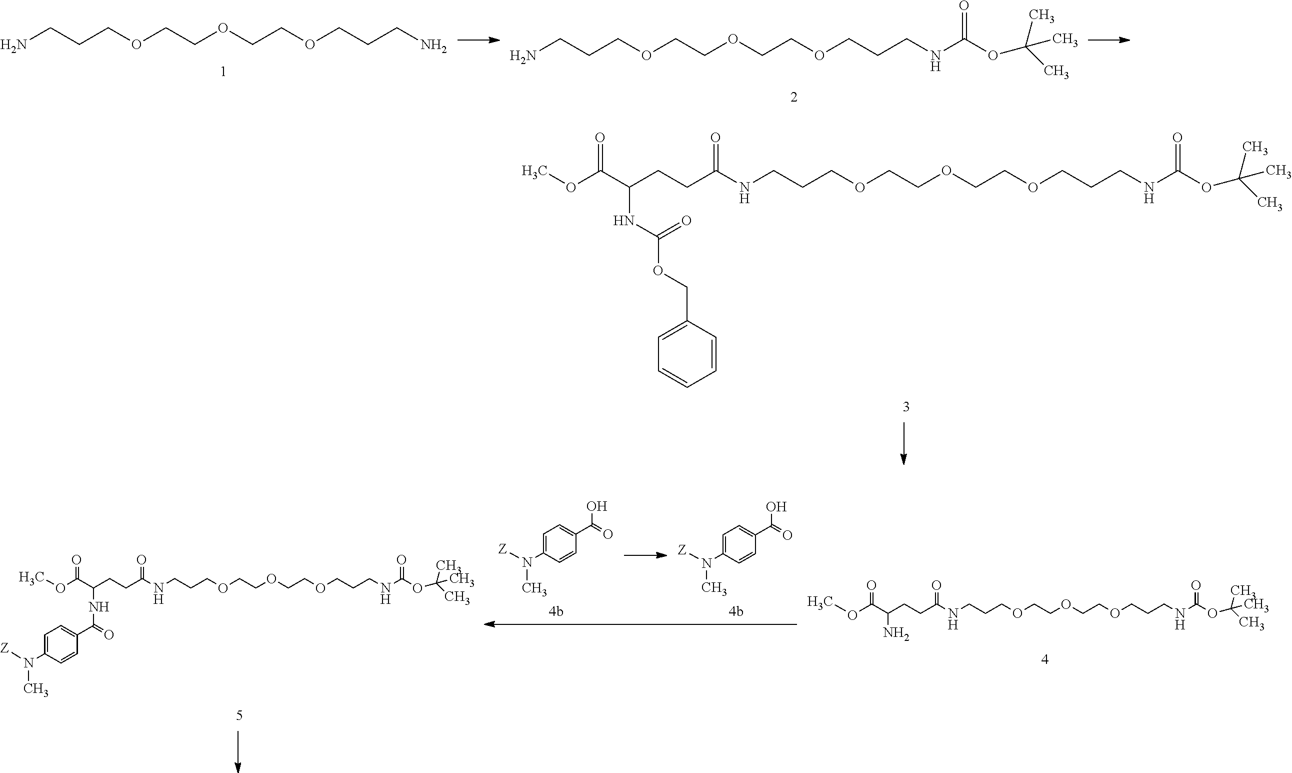

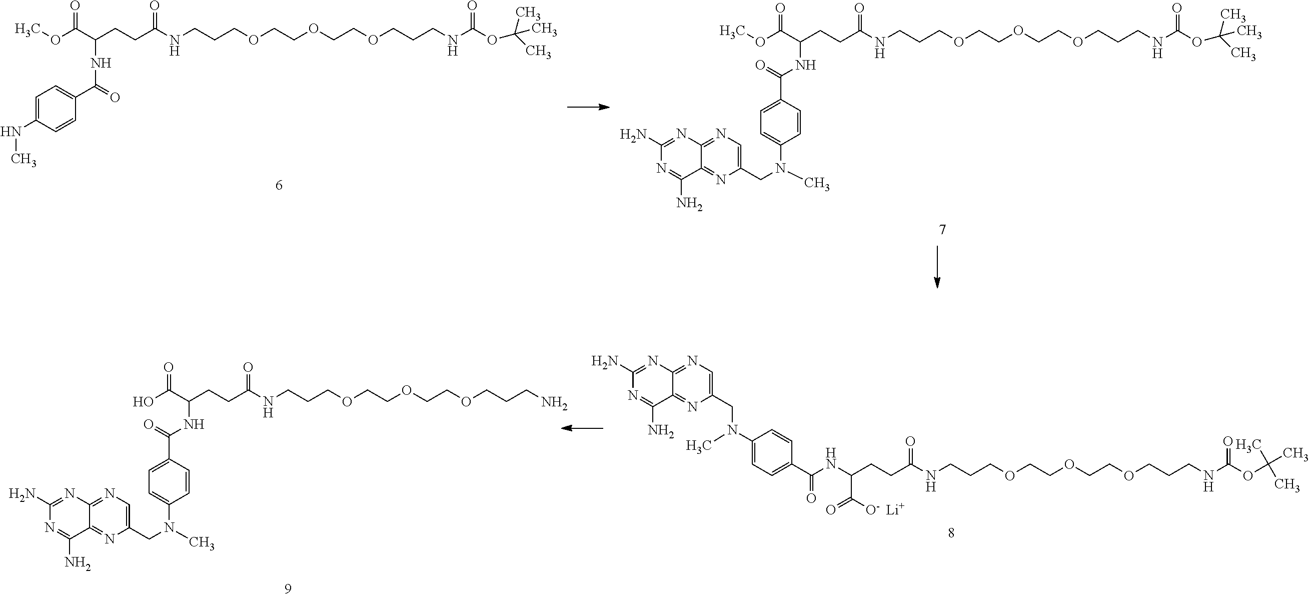 [SMILES: CNc1ccc(C(=O)NC(CCC(=O)NCCCOCCOCCOCCCNC(=O)OC(C)(C)C)C(=O)OC)cc1 ; COC(=O)C(CCC(=O)NCCCOCCOCCOCCCNC(=O)OC(C)(C)C)NC(=O)c1ccc(N(C)Cc2cnc3nc(N)nc(N)c3n2)cc1 ; CN(Cc1cnc2nc(N)nc(N)c2n1)c1ccc(C(=O)NC(CCC(=O)NCCCOCCOCCOCCCN)C(=O)O)cc1 ; CN(Cc1cnc2nc(N)nc(N)c2n1)c1ccc(C(=O)NC(CCC(=O)NCCCOCCOCCOCCCNC(=O)OC(C)(C)C)C(=O)[O-])cc1 ; [Li+]]

The aim of this experiment was to synthesise 50 mg GNP with MTXPEG₃NH₂ (also known as MTX-(EG)₃-NH₂) loading of >12 equivalents per GNP.

The base GNP particle was ([α-GalC2]_{52%}[HSPEG₈COOH]_{48%}@Au), and the coupling was performed by using the EDC/NHS method. Note that in contrast to the positively charged AL of Example 1, the base GNP in this example has PEG₈ (i.e. (EG)₈-containing) ligands with a carboxylic acid terminal functionality (negatively charged) in addition to the α-Gal-C2 ligands. The base GNPs [α-GalC2]_{52%}[HSPEG₈COOH]_{48%}@Au were synthesised essentially as described in WO2017/017063 (see Example 5 thereof), incorporated herein by reference.

Reagents

| Material | Supplier | Batch/R number | Comments |
|---|---|---|---|
| Starting GNP | Midatech | M324-020-01 | [α-GalC2]_{52%}[HSPEG8COOH]_{48%}@Au |
| | Pharma | | |
| EDC | Sigma- | SLBT0569 | — |
| | Aldrich | | |
| NHS | ALDRICH | MKBX1364V | — |
| HEPES | Sigma | SLBM8525V | pH = 7.83, 40 mM |
| MTXPEG3NH₂ | SELVITA | BIO-1323-049-final | started with 60 eq per NP |

Reaction Scheme

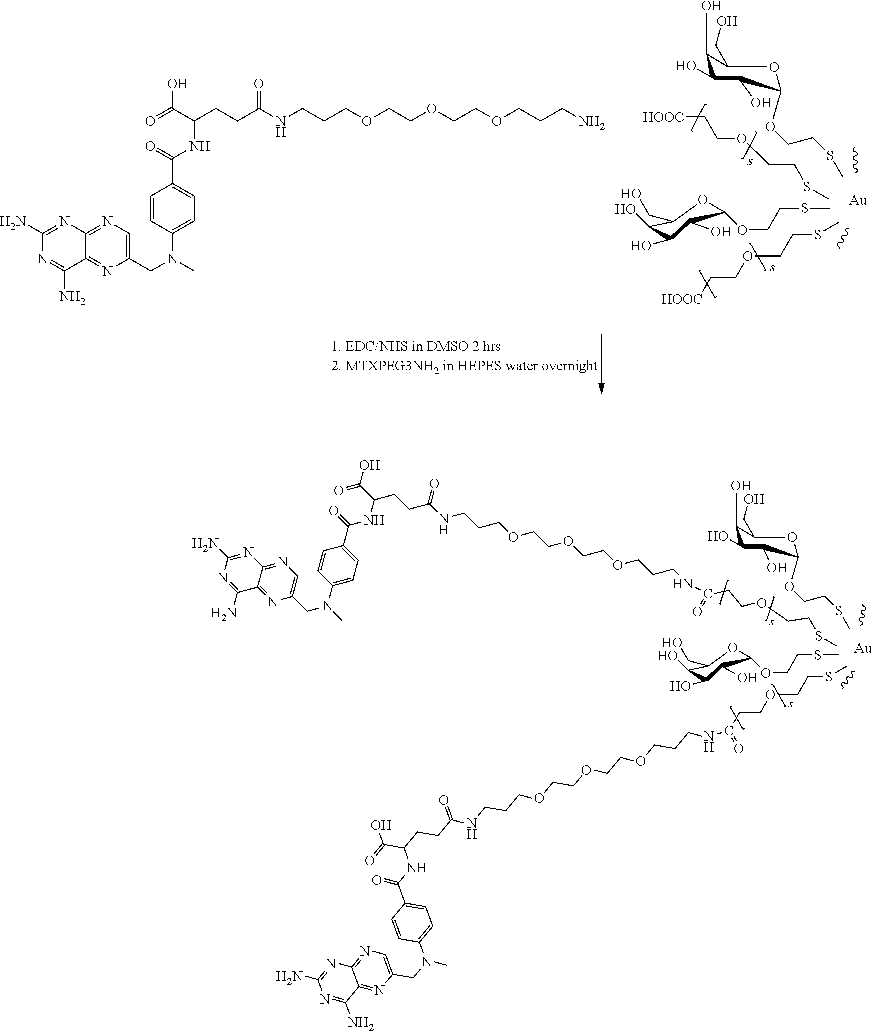

Solvents: 1) 90% DMSO for EDC/NHS activation;
   2) HEPES buffer (pH=7.83) for MTXPEG₃NH₂ coupling.

EDC/NHS Activation

38.12 mg of EDC was dissolved in 3.31 mL DMSO first, then 3.16 mL of this 60 mM EDC DMSO stock was mixed with 43.67 mg of NHS to give a final DMSO stock of EDC (60 mM)/NHS (120 mM).

11 mL 90% DMSO GNP solution (60 mg Au) was kept stirring at 500 rpm, then 2.79 mL of EDC/NHS DMSO stock was added dropwise. The reaction mixture was kept stirring at 500 rpm at R.T for 2 hr ([Au]4.35 mg/mL).

After two hours activation, the GNP-NHS DMSO solution was concentrated in 8×15 mL Amicon tubes (10K) by centrifugation (4300 rpm, 8 min). The GNP final concentration was about 12 mL.

MTXPEG₃NH₂ Coupling:

$$M T X P E {G}_{3} N {H}_{2} ( 60 eq per NP ) : \frac{60 mg Au}{196.97} \div 100 \times 60 \times 656.75 = 120 mg$$

120 mg of MTXPEG₃NH₂ was first dissolved in 20 mL HEPES buffer (pH=7.83), this was then transferred to a 250 mL round bottomed flask. While stirring at 600 rpm at RT (˜22° C.), the 12 mL concentrated GNP-NHS solution was added dropwise. Then, 20 mL of HEPES buffer was added into this mixture. The reaction mixture was stirred at 600 rpm at RT (˜22° C.) overnight ([Au]=1.15 g/L).

The next morning, the reaction solution mixture was concentrated in 15 mL Amicon tubes (10K), and purified by washing with Milli-Q water (×8, 4300 rpm, 8 min per wash). The concentrated solution was then spun at 13.3 G for 5 min (×2) to remove any large size particles from solution. The final concentrated GNP solution was diluted with Milli-Q water to give a final volume of 11 mL.

Chemical and Physical Analysis

| | | Zeta | |
|---|---|---|---|
| | | Potential | |
| [Au] (μg/μl) | Size (nm) | (mV) | UV-VIS |
| 3.889 | 5.678 | −22.8 | No plasmon |
| | | | band at 520 nm |

MTXPEG₃NH₂ content was assessed by Agilent HPLC with the following sample preparation: 8 μg Au was diluted with 0.2M TCEP to give a final volume of 40 μL ([Au]=0.2 g/L), then incubated at 37° C. and agitated at 600 rpm for 1 hr. After incubation, 40 μL of Milli-Q water was added to give a final total volume of 80 μL ([Au]=0.1 g/L). This solution was analysed by HPLC, (20 μL injection→2 μg Au). For MTXPEG₃NH₂ standards: 4 μL of 2 g/L MTXPEG₃NH₂ aqueous stock solution and 36 μL of 0.2M TCEP were incubated at 37° C. and agitated at 600 rpm for 1 hr. To this, 160 μL Milli-Q water was added (total volume=200 μL [MTXPEG₃NH₂]=0.04 g/L). This solution was analysed by HPLC, (10 μL injection→0.4 μg, 20 μL→0.8 μg and 30 μL→1.2 μg).

A standard curve was generated (taking into account the effect of the yellow MTXPEG₃NH₂ compound upon colorimetric gold quantification and thereby correcting the gold concentration). MTXPEG₃NH₂ loading was determined to be 16.7 equivalents per GNP, with incorporation of 97.4%.

In summary, this batch of MTXPEG₃NH₂ particles had the following properties: small size (5.678 nm) with a single size population, negative Zeta potential (−22.8 mV), no plasmon band at 520 nm, MTXPEG₃NH₂ incorporation on GNP was 97.4%, and the loading on the final particles was 16.7 eq per GNP. Consistent results were also found between batches at different reactor sizes (50 mg and 100 mg Au). These results compare favourably to the results obtained in Example 1. In particular, the modified MTX (MTXPEG₃NH₂) facilitated significantly higher loading (16.7 equivalents vs. around 5 equivalents for MTX), high loading efficiency (97.4%) and a single size population. Without being bound by any particular theory, the present inventors consider that the MTXPEG₃NH₂ coupling to the PEG₈COOH ligands of the GNPs avoids the issue of multiple carboxyl sites on MTX described in Example 1 and that this may explain the observed difference between single size distribution/population (Example 2) and two size distributions/populations (Example 1). Moreover, the loading efficiency of 97.4% determined here is markedly higher than even the highest loading efficiency of 83±2% reported in Bessar et al., 2016. The loading of Bessar et al., 2016 in terms of equivalents of MTX per GNP is not reported. However, the weight ratio of Au-3MPS to MTX drug used in the synthesis of Bessar et al., 2016 was 5:1 (i.e. excess of GNPs). In conclusion, the [α-GalC2][MTXPEG₃NH—CO-PEG₈]@Au GNPs exhibit high MTX loading and suitable physical properties for skin penetration.

### Example 3—Formulation of [α-GalC2][MTXPEG₃NH—CO-PEG]@Au GNPs into Hydrogels

Currently available marketed topical formulations of methotrexate exhibit poor penetration through the stratum corneum due to the hydrosoluble nature of the drug, which is mostly in a dissociated form at physiological pH (pH 6). The ultra-small size (<5 nm) of the GNPs disclosed herein having a corona comprising carbohydrate ligands, which allows for suitable net surface charge, may offer potential for increasing the capacity of methotrexate penetration across intact skin.

Recently, a topical gold nanoparticle cream formulation was reported by Bessar et al. 2016 to show preliminary proof of percutaneous adsorption of methotrexate conjugated GNP. Hydrogels have also been applied for the development of topical nanoparticle formulations, as these provide a single-phase vehicle that could allow greater flexibility and control of drug delivery from the formulation. In addition, hydrogels offer the advantage of rapid evaporation leaving no residual formulation on the skin compared to commercially available ointments, in which high affinity between drug and formulation base compromises efficient drug transfer into the skin. Therefore, Carbopol hydrogels were selected for the development of GNP based topical formulations.

The following polymers (Lubrizol Corporation) were evaluated: Carbopol® ETD 2020 (C10-30 alkyl acrylate cross polymer), Carbopol® 980 NF polymer and Carbopol® 974P NF Polymer. Gels were prepared by dispersing 1-3% w/v of Carbopol polymer (w/v) into purified water with constant mixing and thus allowed to hydrate for 5 hours. Care was taken to avoid air entrapment by agitating the solution slowly on a rocker during preparation of the gel. After 5 hours, the pH of the gel was adjusted to pH 7.4 using triethylamine (Sigma-Aldrich, Lot #STBF616V) to neutralise the pH and turn the solution into a gel (triethanolamine is contemplated herein as a suitable alternative to triethylamine). A 2% Carbopol® 980 gel was found to produce a clear, homogenous gel whereas ETD 2020 gel was more difficult to produce homogeneity. Therefore, formulation of the gold glyconanoparticles into a hydrogel proceeded with the Carbopol® 980 NF polymer.

MTXPEG₃NH₂-loaded GNPs were prepared essentially as described in Example 2. For production of methotrexate-GNP hydrogel, 2% w/v Carbopol®980 was initially dispersed for 5 hours with constant mixing. The MTX-PEG₃-NH₂-loaded GNPs were concentrated using Amicon centrifugal filter tubes (10 K membrane molecular weight cut-off) with centrifugation at 5000 rpm for 10 min. Prior to addition to the 2% Carbopol®980 solution, the pH of MTX-PEG₃-NH₂-loaded GNPs was adjusted to pH 2.6. The acidic MTX-PEG₃-NH₂-loaded GNPs were then added to the 2% Carbopol®980 solution. However, the nanoparticles were observed to precipitate rapidly in the Carbopol®980 solution. Plain methotrexate drug gel was prepared by dissolving MTX-PEG₃-NH₂ in water and adjusting the pH to pH 4.5. The MTX-PEG₃-NH₂ solution was added to the previously made 2% Carbopol®980 solution. However, a small level of yellow precipitation was also observed.

The method for formulating gold nanoparticles into Carbopol®980 gels was optimised by testing the effects of pH and speed of addition of nanoparticles using control [α-Gal][PEG₈COOH]@Au GNPs. Homogenous nanoparticle gels without precipitation were obtained when the pH of the Carbopol®980 solution was adjusted to pH 7.4 prior to the drop-wise addition of the [α-Gal][PEG₈COOH]@Au GNPs with constant mixing. Similarly, for methotrexate gel (without nanoparticles), a homogenous yellow gel without precipitation was obtained when the pH of the Carbopol®980 solution was adjusted to pH 7.4 prior to the drop-wise addition of modified methotrexate. The gels were all stored at 4° C.

For production of methotrexate-GNP hydrogel, 2% w/v Carbopol®980 was dispersed for 5 hours with constant mixing. The pH of the Carbopol®980 solution was adjusted to pH 7.4 to produce a clear gel. MTX-PEG₃-NH₂-loaded GNPs were concentrated using Amicon centrifugal filter tubes and then added to the 2% Carbopol®980 gel. The resulting MTX-PEG₃-NH₂-loaded GNP hydrogel was a homogeneous brown gel, with no precipitation of MTX-PEG₃-NH₂-loaded GNPs observed in the gel. Control GNP (no drug) gel was also prepared using [α-Gal-C2][PEG₈COOH]@Au GNPs and found to produce a brown, homogenous gel. Plain methotrexate drug gel was prepared by adding MTX-PEG₃-NH₂ dissolved in water to the pH 7.4 adjusted Carbopol®980 gel (2%). The methotrexate was found to be incorporated readily, producing a yellow homogenous hydrogel, with no precipitation of the methotrexate derivative observed.

The concentration of MTX-PEG₃-NH₂ in the MTX-PEG₃-NH₂-loaded GNP hydrogel was in the range 0.18-0.2% (w/w).

MTX concentration in previously reported topical formulations are generally in the range 0.25% to 0.5% (see, e.g., Lakshmi et al., Indian J Dermatol Venereol Leprol, 2007, Vol. 73, pp. 157-161 and Jabur et al., J Fac Med Baghdad, 2010, Vol. 52, No. 1, pp. 32-36).

The GNP (+/−MTX-PEG₃-NH₂) hydrogel formulations together with MTX-PEG₃-NH₂ hydrogel (i.e. no GNPs) and Carbopol®980 hydrogel formulations were utilised for in vivo testing to determine the efficacy of topical applied GNP (+/−MTX-PEG₃-NH₂) hydrogel formulations in enhancing delivery of methotrexate into inflamed skin in the imiquimod-induced psoriasis-like inflammation mouse model (see Example 4 below).

### Example 4—MTX-PEG₃-NH₂-Loaded GNPs Tested in an Imiquimod (IMQ)-Induced Psoriatic Mouse Model

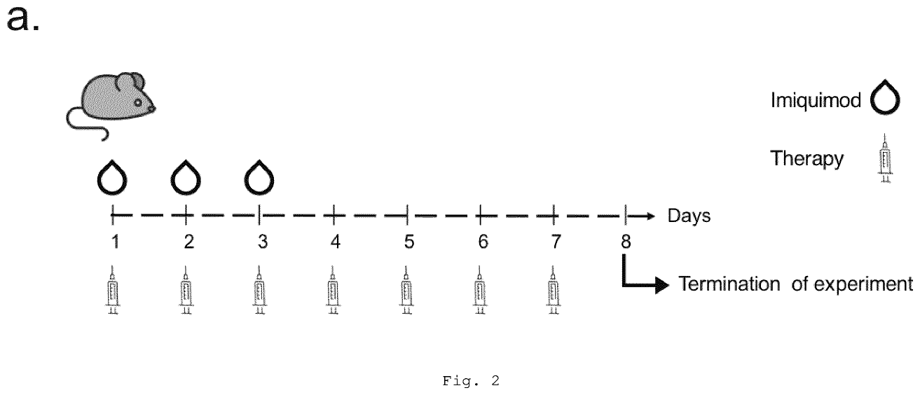

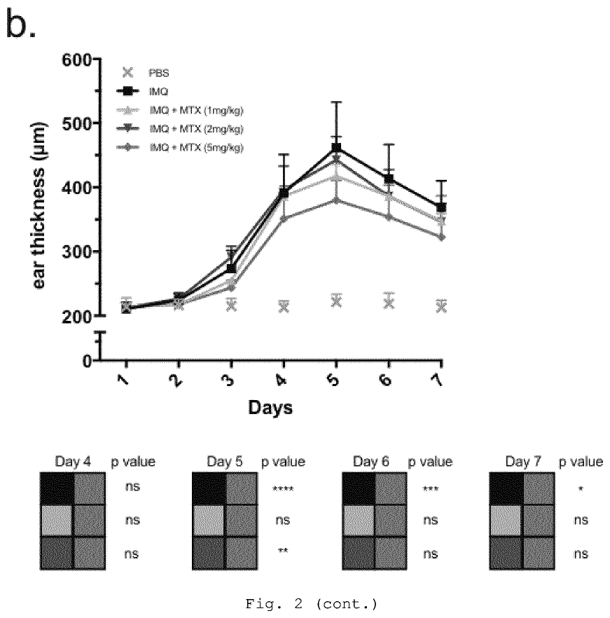

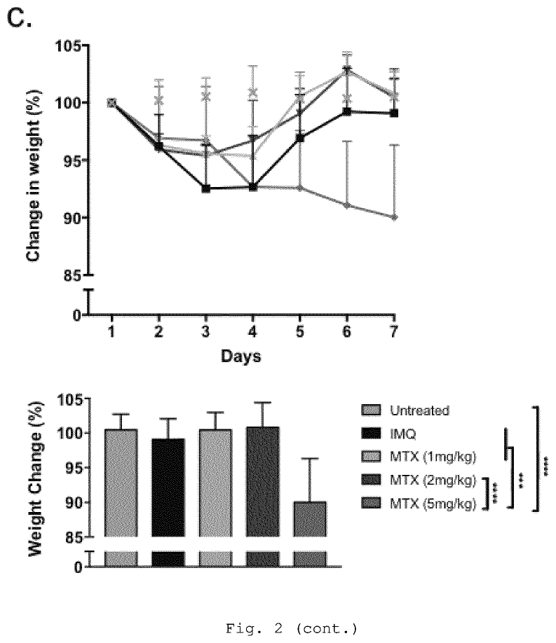

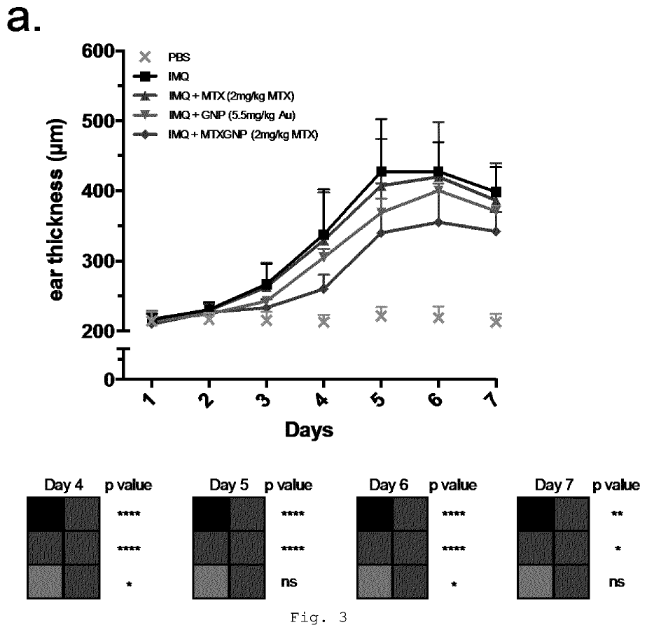

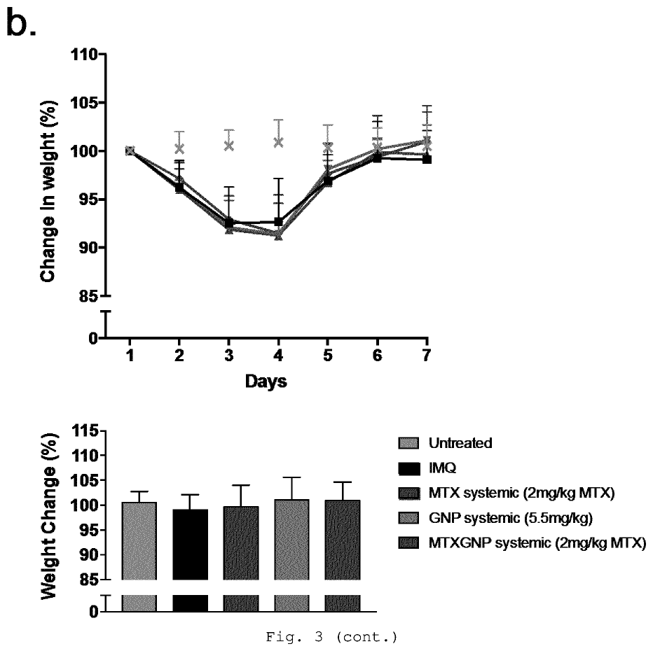

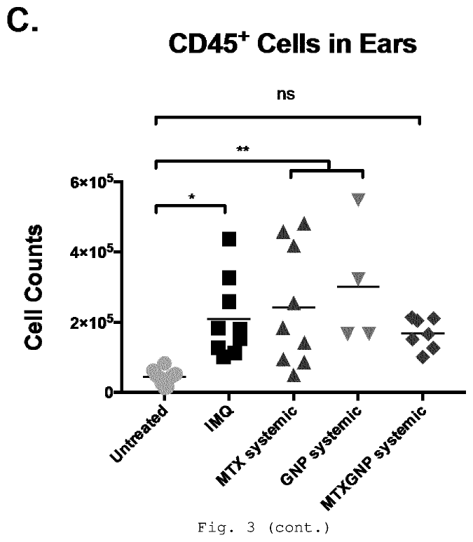

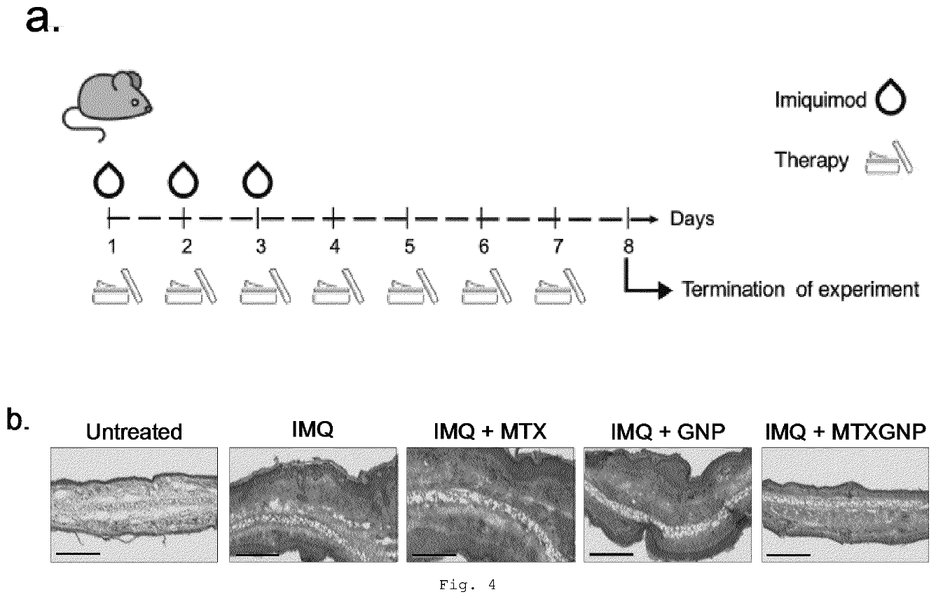

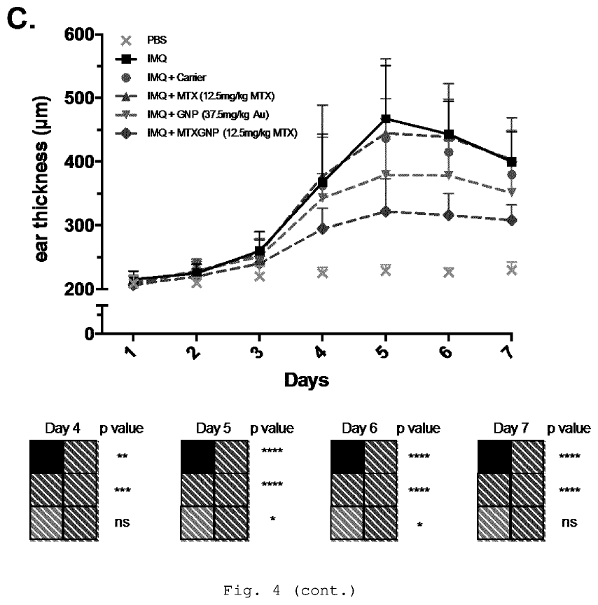

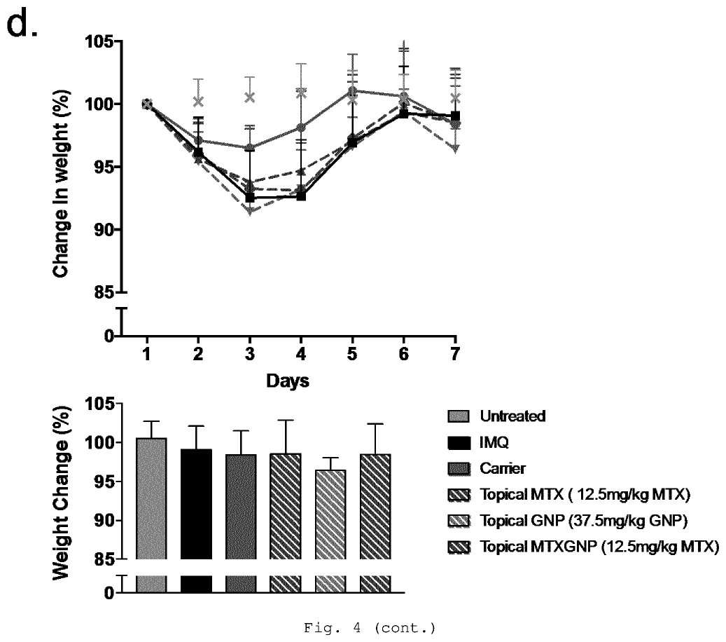

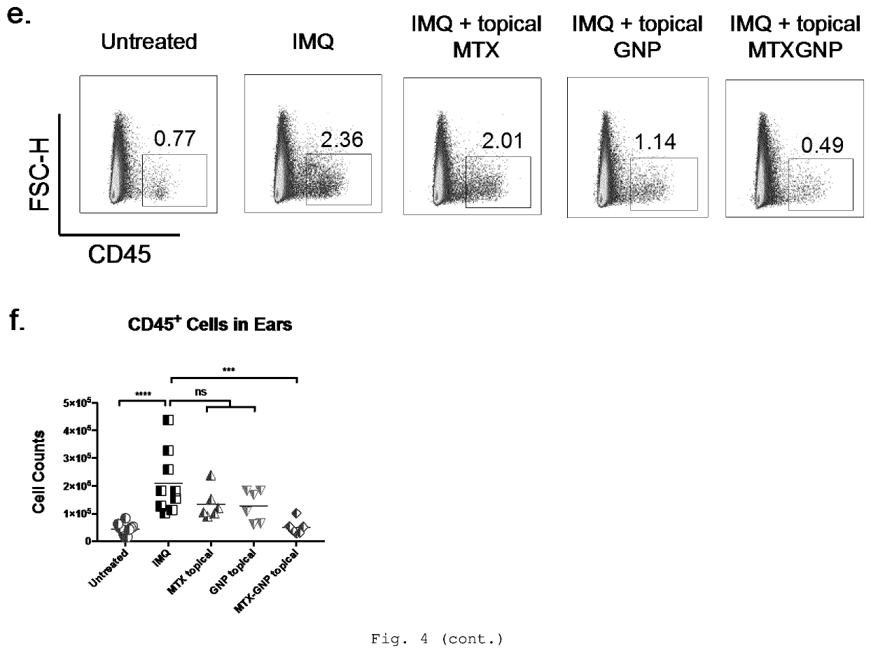

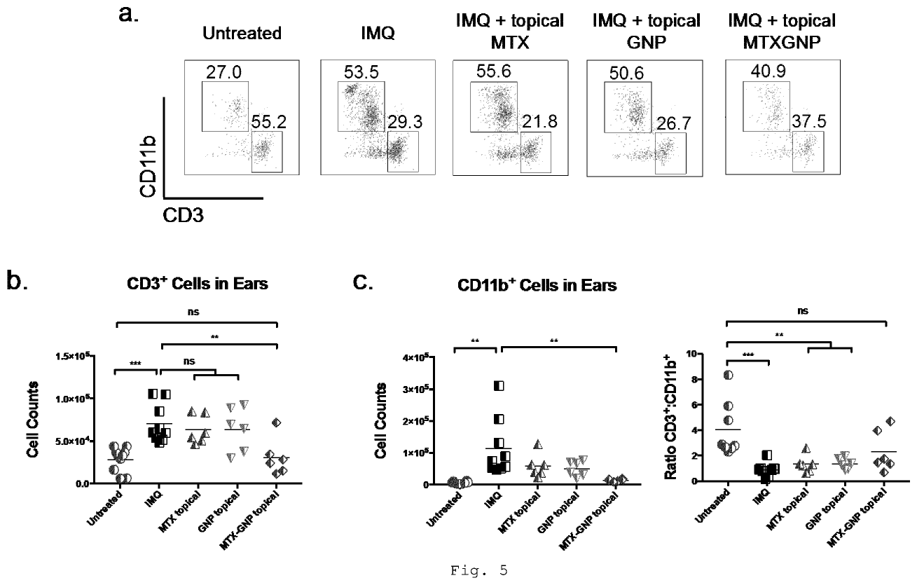

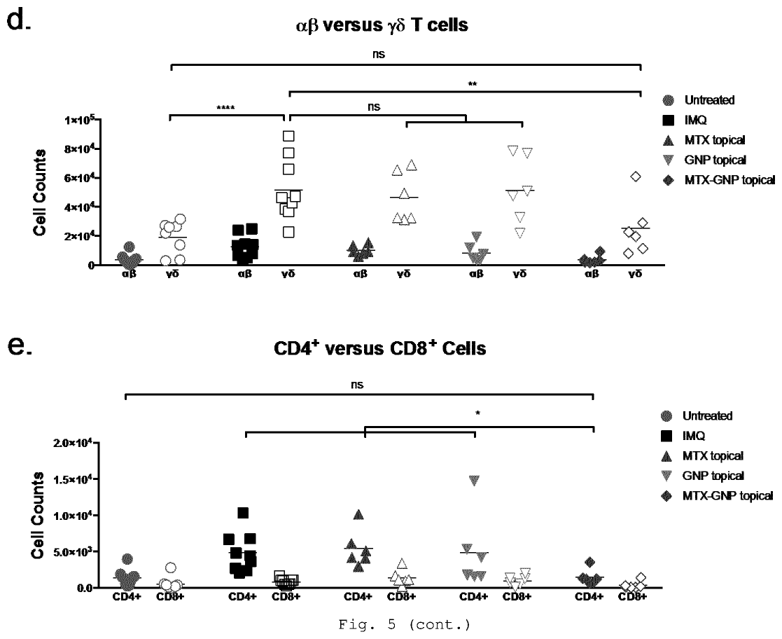

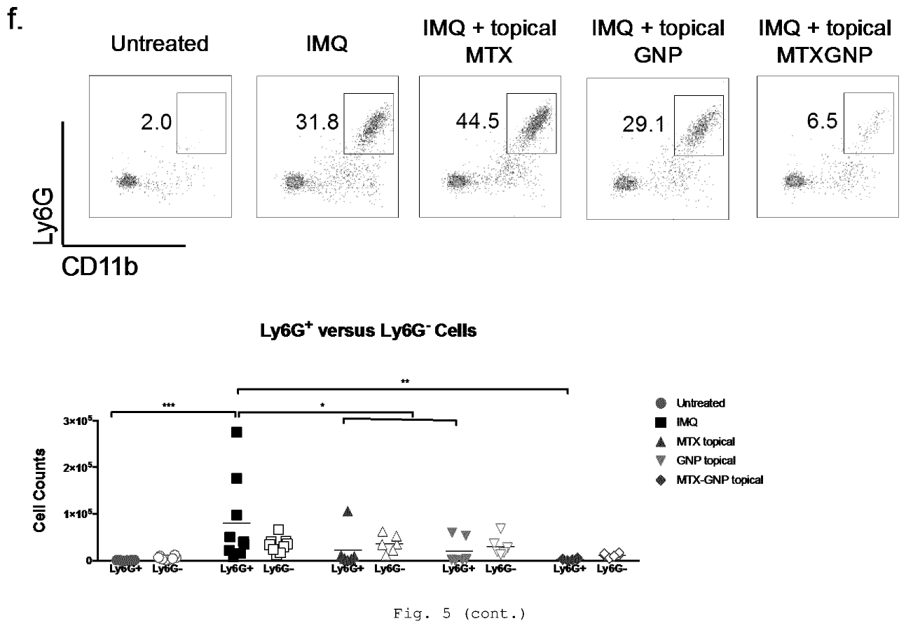

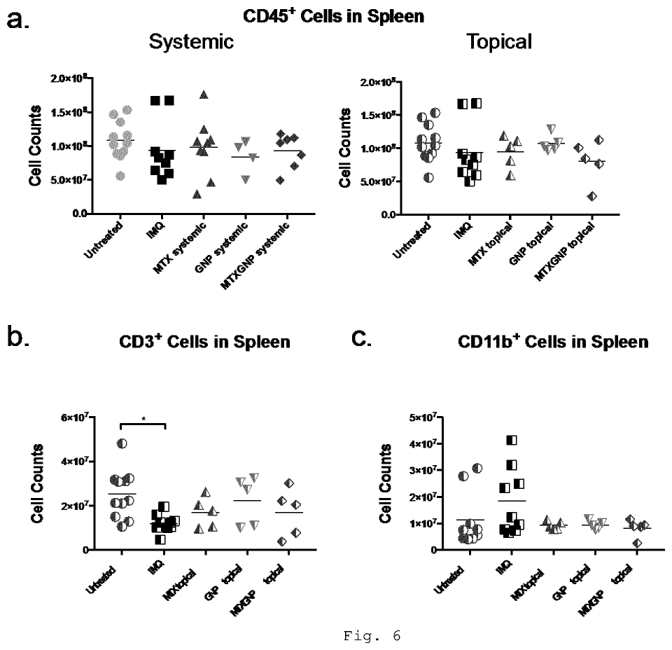

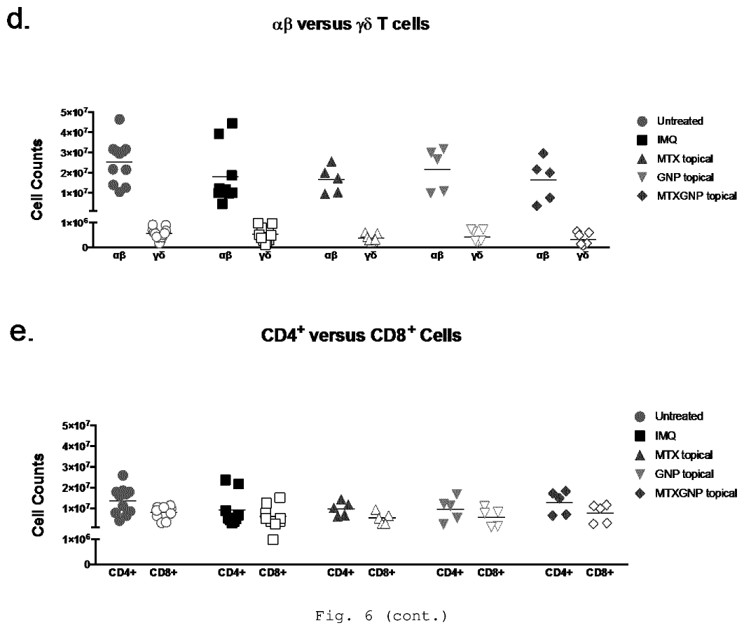

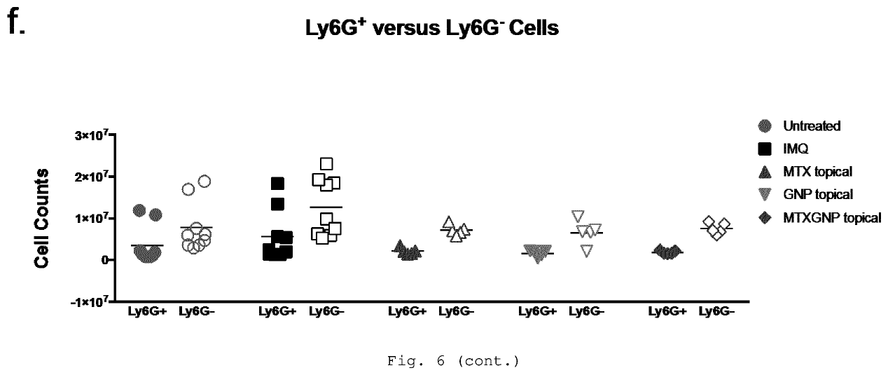

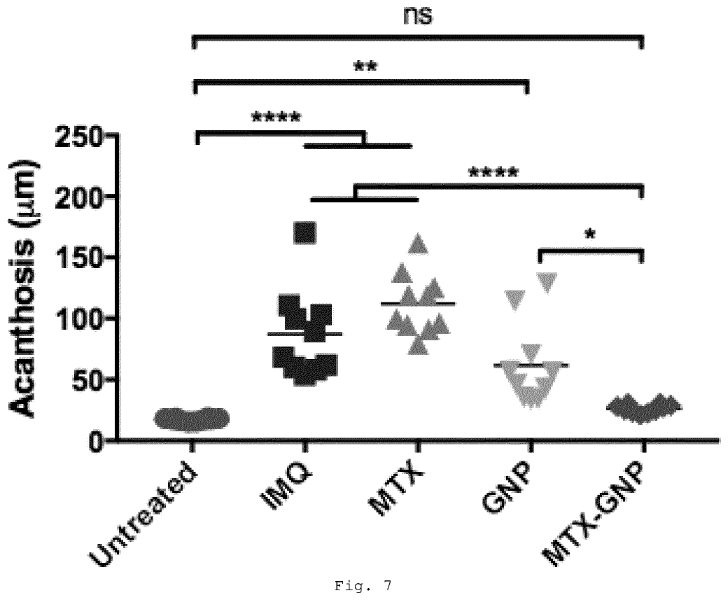

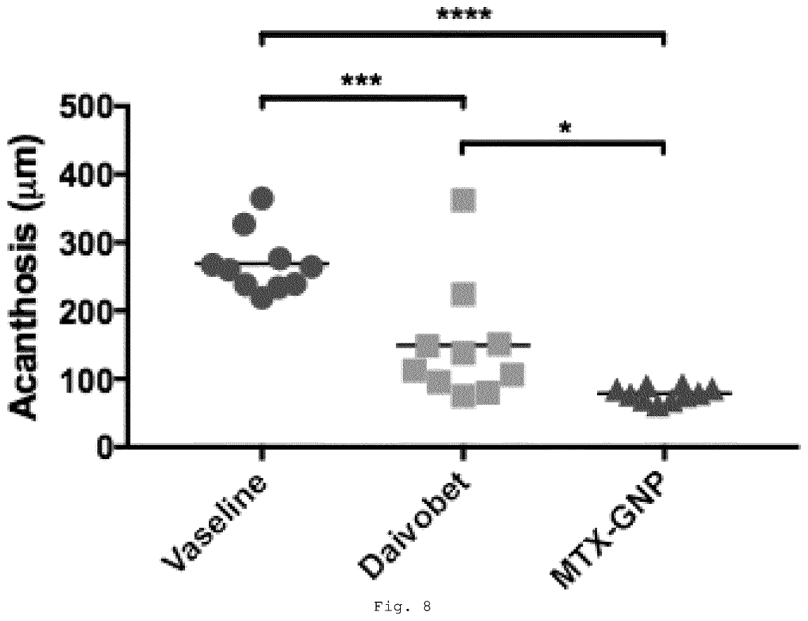

The aim of this study was to assess the therapeutic efficacy of the MTX-GNP gel formulation (MTX-PEG₃-NH₂-loaded GNPs formulated as a hydrogel as described in Examples 2 and 3) having enhanced skin permeability, using the Imiquimod (IMQ)-induced mouse model of psoriasis, where IMQ is applied on the ears of a mouse for three consecutive days (FIG. 2(a)). Clinical efficacy and in vivo tolerability were assessed against relevant controls for both systemic (subcutaneous injection) and topical administration.

First, we evaluated tolerance of mice to MTX with a dose escalation setup (FIG. 2b). It was found that a clinically effective treatment with MTX (5 mg/kg daily) impairs animal constitution, leading to an uncontrollable continuous weight loss throughout therapy (FIG. 2c). A tolerable dose of the drug (2 mg/kg daily) on the other hand demonstrated no significant inflammation control, resulting in ear thickness measures comparable to the IMQ alone control group (FIG. 2b). These results underline the drawbacks of systemic administration of free MTX as a therapy for psoriasis.

Subcutaneous systemic administration of MTX-PEG₃-NH₂-loaded GNPs comprising the same 2 mg/kg dose of MTX that was ineffective but tolerable when given as the free drug, significantly ameliorated IMQ-induced inflammation, indicating an additive anti-inflammatory action of GNPs (see FIG. 3c). The treatment regimen with the MTX-PEG₃-NH₂-loaded GNPs led to no significant systemic toxicity, assessed by liver enzyme measurements (data not shown) and daily weight monitoring (FIG. 3b). These results surprisingly show that MTX-PEG₃-NH₂-loaded GNPs even when administered systemically exhibit improved efficacy and tolerability relative to MTX alone.

Topical MTX-PEG₃-NH₂-loaded GNPs were formulated as a hydrogel (Example 3) based on prior evaluation of clinical efficacy and optimal tolerance to systemic administration of MTX in IMQ model. The topical experimental scheme is shown in FIG. 4a. Daily topical application of gel-based MTX-PEG₃-NH₂ GNPs drastically reduced the ear inflammation induced by IMQ (FIG. 4b). Three-day IMQ treatment led to a vigorous thickening of the ears, which was significantly prevented by the topical gel-based MTX-PEG₃-NH₂-loaded GNP therapy (FIG. 4c). A gel-based MTX-PEG₃-NH₂ formulation, prepared under same synthesis conditions for the MTX-PEG₃-NH₂-loaded GNP gel, did not affect the ear thickness induced upon IMQ (FIG. 4c). The relative lack of effect of the MTX-PEG₃-NH₂ gel formulation is presumably due to the previously described hydrophilic nature and poor skin penetration of MTX. Interestingly, a gel formulated with GNPs alone (i.e. without MTX) also caused a modest but significant reduction of ear thickness (FIG. 4c), which can possibly be attributed to reported anti-inflammatory effect of GNPs (Shukla, R. et al. Langmuir 21, 10644-10654, doi:10.1021/1a0513712 (2005), Tsai, C. Y. et al. J Immunol 188, 68-76, doi:10.4049/jimmunol.1100344 (2012) and Moyano, D. F. et al. Chem 1, 320-327, doi:10.1016/j.chempr.2016.07.007 (2016)).

A clear histological difference was observed on IMQ-treated ears under topical MTX-PEG₃-NH₂-loaded GNP gel therapy versus MTX-PEG₃-NH₂ gel or GNP gel therapy alone and controls (FIG. 4b). Topical administration of MTX-PEG₃-NH₂-loaded GNP gel was tolerated well by the animals, and no significant systemic toxicity was observed, assessed by liver enzyme measurements (data not shown) and daily weight monitoring (FIG. 4d).

Therefore, these results show that localized topical therapy with MTX-PEG₃-NH₂-loaded GNP gel can counteract IMQ-induced inflammation with minimal to no interference with animal wellbeing.

Further analysis was conducted on the inflammatory milieu in the IMQ-treated ears of the mice receiving the aforementioned therapies using fluorescence-activated cell sorting (FACS) analysis. Mice receiving topical therapy with MTX-PEG₃-NH₂-loaded GNP gel demonstrated significantly lower number of infiltrating immune cells into the ears, indicated by low counts of CD45⁺ cells, compared to the IMQ alone group. All other tested topical therapies (IMQ+MTX, IMQ+GNP) had comparable immune infiltration to IMQ-alone group (FIGS. 4 and 5). In particular, the MTX-PEG₃-NH₂-loaded GNP gel treated group showed restoration of the balance between CD11b⁺ and CD3⁺ T cells (FIG. 5c) A more detailed FACS analysis was performed to pinpoint the impact of topical therapy with MTX-PEG₃-NH₂-loaded GNP gel on key adaptive and innate players of psoriatic inflammation. We have demonstrated that localized topical treatment with MTX-PEG₃-NH₂-loaded GNP gel can substantially clear the skin of γδ T cells and Ly6G⁺ neutrophils, and significantly limit CD4⁺ αβ T cells whereas the CD8⁺ T cells remain unaffected (FIGS. 5d and e). No apparent systemic effect on immune populations in the spleen was observed (FIG. 6).

Conclusion

The present results show that the MTX-loaded GNP formulation of the present invention is skin-penetrating and alleviates skin inflammation upon topical application. Topical MTX-GNP gel formulations were able to overcome imiquimod-induced inflammation, reducing it close to baseline and also reduce neutrophils equivalent to baseline. Furthermore, localized MTX-GNP application was well tolerated by the animals, unlike the systemic MTX administration, which at high doses irrevocably impaired animal wellbeing. The key players of psoriasis including γδ T cells, neutrophils and CD4⁺ αβ T cells are not significantly proliferating compared to untreated controls in the MTX-GNP treated groups. Given its strong anti-inflammatory capacity and tolerability, gel-based MTX-GNPs, including MTX-PEG₃-NH₂-loaded GNPs potentially offer an attractive alternative non-steroidal topical therapeutic option for psoriasis and even a broader range of inflammatory skin diseases. Indeed, the inventors consider the following skin disorders to be disorders expected to benefit from treatment with the nanoparticle formulations of the present invention: psoriasis (e.g. psoriasis vulgaris or pustular, inverse, napkin, nail, guttate, oral, or seborrheic-like psoriasis). In some embodiments the disorder may be selected from: Pityriasis rubra pilaris, cutaneous lichen, rosacea, alopecia areata, cutaneous lymphoma, an eczematous skin disorder (such as atopic dermatitis, cutaneous drug reaction, prurigo nodularis, or cutaneous mastocytosis), an autoimmune bullous skin disorder (such as pemphigus/pemphigoid, dermatitis herpetiformis, epidermolysis bullosa), cutaneous lupus, cutaneous vasculitis, Behcet's disease, sclerodermiform skin disease, a neutrophil mediated skin disease (such as pyoderma gangrenosum, sweet syndrome, hidradenitis suppurativa, SAPHO syndrome), a granulomatous skin disease (such as granuloma annulare, erythema annulare, erythema nodosum, sarcoidosis or necrobiosis lipoidica).

### Example 5—Comparison of MTX-PEG₃-NH₂-Loaded GNPs Carbopol Hydrogel with Daivobet Gel (Psoriasis Topical Standard of Care) in a Xenotransplantion Human Skin AGR129 Mouse Model

Boyman et al., J. Exp. Med., 2004, Vol. 199, No. 5, pp. 731-736 describe an animal model in which skin lesions spontaneously developed when symptomless pre-psoriatic human skin was engrafted onto AGR129 mice, deficient in type I and type II interferon receptors and for the recombination activating gene 2. Upon engraftment, resident human T cells in pre-psoriatic skin underwent local proliferation. T cell proliferation was crucial for development of a psoriatic phenotype because blocking of T cells led to inhibition of psoriasis development. Tumor necrosis factor-α was a key regulator of local T cell proliferation and subsequent disease development. The Boyman et al., 2004 AGR129 mouse model represents a highly relevant model system for the investigation of potential psoriasis therapies. In particular, this model provides a means to study effects on human skin, including the ability of a test compound to inhibit the development of psoriasis, and therefore offers additionally relevant features to the imiquimod-treated mouse model described in Example 4.

Methodology

Keratome biopsies of non-symptomatic skin were obtained from human psoriasis patients. A skin sample (1 cm²) was then grafted onto the shaved back of the AGR129 mouse. AGR129 mice are deficient in type I (A) and type 2 (G) interferon receptors, and they are also RAG-2^{KO} (R). Hence they lack, T and B cells, and the NK cells are non-functional. This specific background ensures graft acceptance.

Transplanted non-lesional skin developed into a psoriatic phenotype within 4-6 weeks. One aim of the present study was to investigate the ability of the MTX-PEG₃-NH₂-loaded GNP hydrogel topical formulation to block this development of psoriatic phenotype, and to see how the MTX-PEG₃-NH₂-loaded GNP hydrogel performs in comparison with a standard topical treatment Daivobet gel, which contains betamethasone and calcipotriol. In addition to the MTX-PEG₃-NH₂-loaded GNP hydrogel treatment group, Vaseline and Daivobet gel control groups were included.

Daily topical treatments began 21 days after transplantation for 2 weeks. 10-12 mice were transplanted per experiment. Animals were sacrificed on day 35. The immune composition of the graft was determined by histology and FACS.

Results

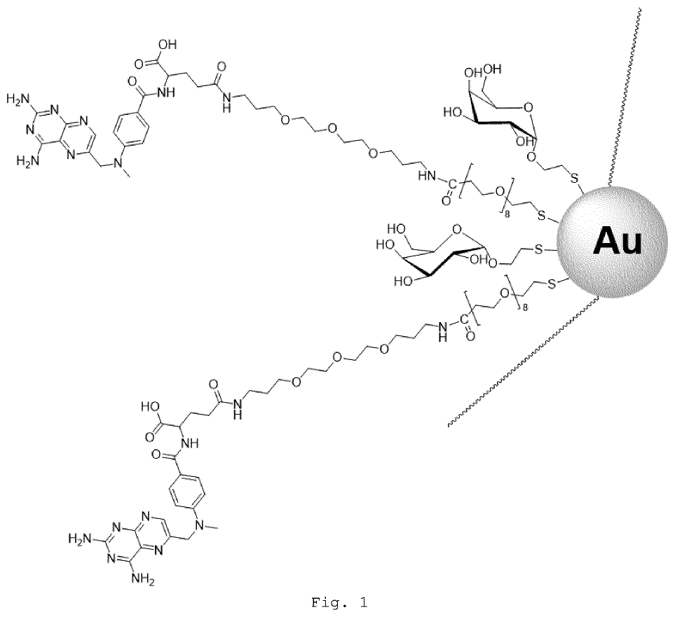

Maximal epidermal thickness (acanthosis) was measured from the junction of the stratum corneum and viable epidermis (stratum granulosum or stratum spinosum) to the deepest portion of the rete ridge (as shown in FIG. 1 of Fraki et al., Journal of Investigative Dermatology, 1983, Vol. 80, No. 6, Suppl. 1, pp. 31s-35s, incorporated herein by reference). Measurements were made using the ImageScope program. Ten consecutive retes were measured and the mean expressed in micrometers as the epidermal thickness (FIG. 8).

As shown in FIG. 8, it was found that the results for the MTX-PEG₃-NH₂-loaded GNP hydrogel were highly reproducible and demonstrated that the MTX-PEG₃-NH₂-loaded GNP hydrogel inhibited the development of psoriasis compared to both Vaseline control (P<0.0001) and Daivobet (P<0.05). These results therefore show evidence of the ability of the MTX-loaded nanoparticles of the present invention to significantly inhibit the onset or development of psoriasis in a sophisticated in vivo model, employing human pre-psoriatic skin.

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

1. A nanoparticle comprising:
a core comprising a metal and/or a semiconductor; and
a plurality of ligands covalently linked to the core, wherein said ligands comprise:
(i) at least one dilution ligand comprising a carbohydrate, glutathione or a polyethyleneglycol moiety; and
(ii) a ligand of the formula MTX-L-, wherein MTX-L-represents methotrexate coupled to said core via a linker L.

2. The nanoparticle of claim 1, wherein L comprises a linear chain of 2 to 100 atoms in length between the methotrexate and the core.

3. The nanoparticle of claim 1 or claim 2, wherein L comprises a group —(CH₂)ₙ— and/or —(OCH₂CH₂)ₘ—, wherein n and m are independently 1.

4. The nanoparticle of any one of the preceding claims, wherein L is of the formula: L₁-Z-L₂
wherein L₁ comprises a first linker portion comprising a C2-C12 glycol and/or C1-C12 alkyl chain, L₂ comprises a second linker portion comprising a C2-C12 glycol and/or C1-C12 alkyl chain, wherein L₁ and L₂ may be the same or different, and wherein Z represents a divalent linker group of up to 10 atoms linking L₁ and L₂ and Z comprises at least 2 heteroatoms.

5. The nanoparticle of claim 4, wherein Z comprises a 3-10 membered carboaromatic, a 3-10 membered carbocycle, a 3-10 membered heterocycle, a 3-10 membered heteroaromatic, an imide, an amidine, a guanidine, a 1,2,3-triazole, a sulfoxide, a sulfone, a thioester, a thioamide, a thiourea, an amide, an ester, a carbamate, a carbonate ester or a urea.

6. The nanoparticle of claim 4 or claim 5, wherein L₁ comprises —(OCH₂CH₂)ₚ— and L_{z} comprises —(OCH₂CH₂)_{q}— and wherein each of p and q is a number in the range 2 to 10, and wherein p and q may be the same or different.

7. The nanoparticle of any one of the preceding claims, wherein MTX-L- is of the formula:

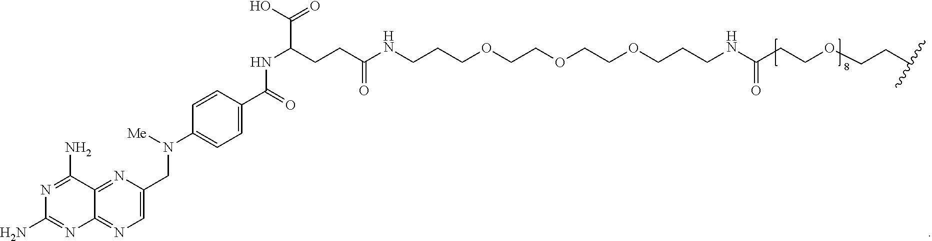

8. The nanoparticle of any one of claims 1 to 6, wherein MTX-L- is of the formula:

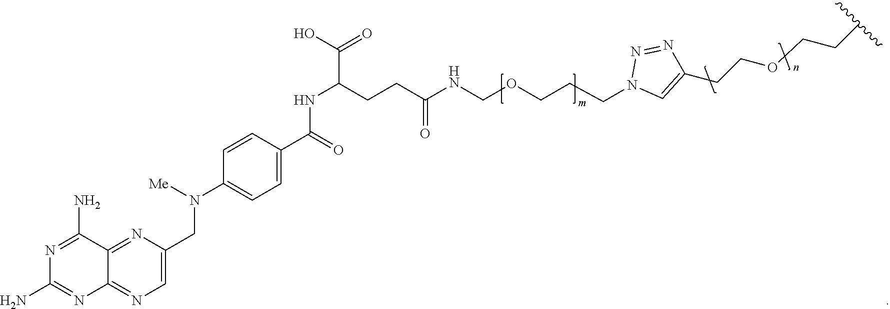

9. The nanoparticle of any one of the claims 1 to 6, wherein MTX-L- is of the formula:

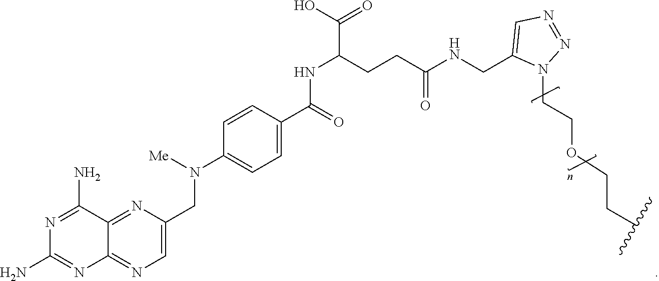

10. The nanoparticle of any one of claims 1 to 6, wherein MTX-L- is of the formula:

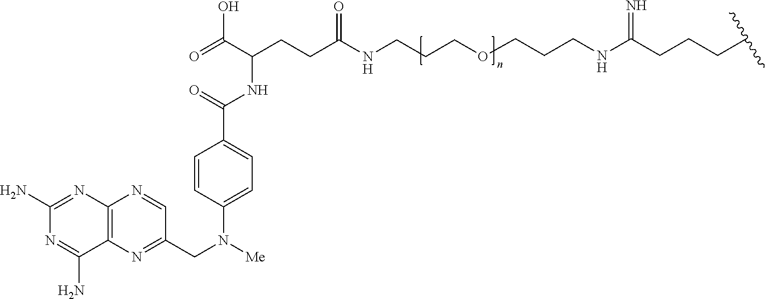

11. The nanoparticle of any one of claims 1 to 6, wherein MTX-L- is of the formula:

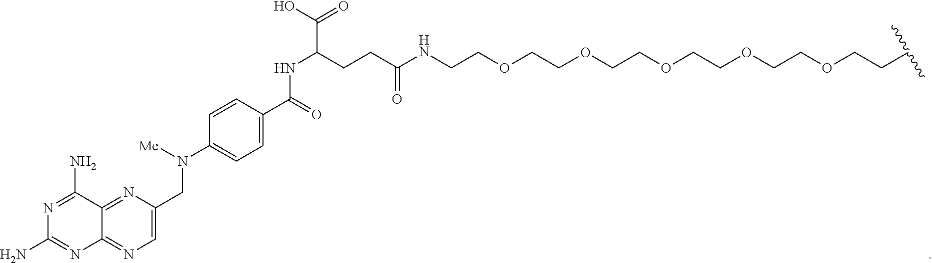 [SMILES: *CCOCCOCCOCCOCCOCCNC(=O)CCC(NC(=O)c1ccc(N(C)Cc2cnc3nc(N)nc(N)c3n2)cc1)C(=O)O]

12. The nanoparticle of any one of the preceding claims, wherein L is bound to the core via a terminal sulphur atom.

13. The nanoparticle of any one of the preceding claims, wherein said dilution ligand comprises a carbohydrate which is a monosaccharide or a disaccharide.

14. The nanoparticle of claim 13, wherein said dilution ligand comprises galactose, glucose, mannose, fucose, maltose, lactose, galactosamine and/or N-acetylglucosamine.

15. The nanoparticle of claim 13 or claim 14, wherein said dilution ligand comprises 2′-thioethyl-α-D-galactopyranoside or 2′-thioethyl-β-D-glucopyranoside.

16. The nanoparticle of any one of the preceding claims, wherein the core comprises a metal selected from the group consisting of: Au, Ag, Cu, Pt, Pd, Fe, Co, Gd, Zn or any combination thereof.

17. The nanoparticle of claim 16, wherein the core comprises gold.

18. The nanoparticle of any one of the preceding claims, wherein the diameter of the core is in the range 1 nm to 5 nm.

19. The nanoparticle of any one of the preceding claims, wherein the diameter of the nanoparticle including its ligands is in the range 3 nm to 50 nm.

20. The nanoparticle of any one of the preceding claims wherein the total number of ligands per core is in the range 20 to 200.

21. The nanoparticle of any one of the preceding claims, wherein the number of ligands of said formula MTX-L- per core is at least 3, such as in the range 3 to 100 per core.

22. The nanoparticle of claim 21, wherein the number of ligands of said formula MTX-L- per core is at least 3, such as in the range 5-10, 10-15 or 15-20 per core.

23. A nanoparticle according to claim 1 having the following structure: 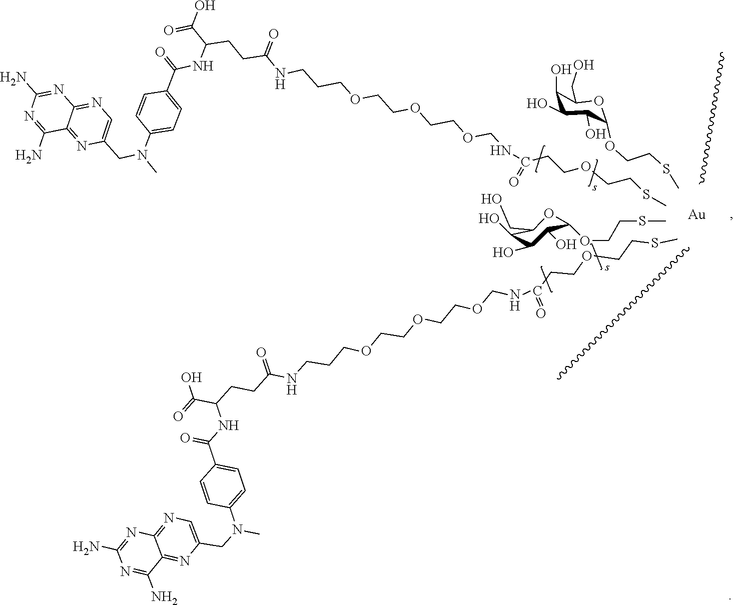
wherein the total number of ligands per core is at least 5, and the total number of methotrexate-containing ligands per core is at least 3.

24. A nanoparticle according to claim 1 having the following structure: 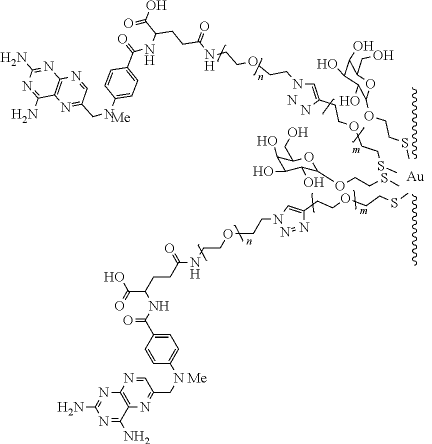
wherein n and m are independently 1, 2, 3, 4, 5, 6, 7, 8, 9 or 10, the total number of ligands per core is at least 5, and the total number of methotrexate-containing ligands per core is at least 3.

25. A nanoparticle according to claim 1 having the following structure: 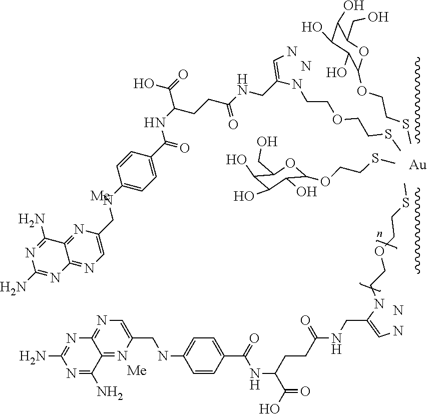
wherein n is an integer of between 1 and 15, the total number of ligands per core is at least 5, and the total number of methotrexate-containing ligands per core is at least 3.

26. A nanoparticle according to claim 1 having the following structure: 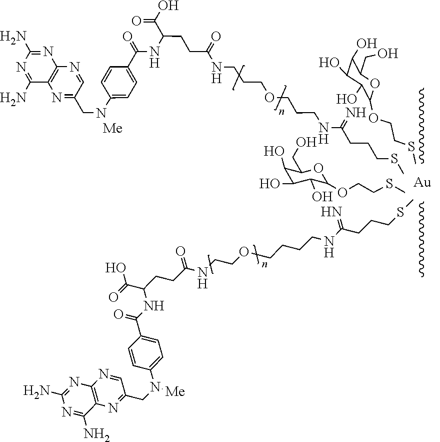
wherein n is an integer of between 1 and 15, the total number of ligands per core is at least 5, and the total number of methotrexate-containing ligands per core is at least 3.

27. A pharmaceutical composition comprising a plurality of nanoparticles of any one of the preceding claims and at least one pharmaceutically acceptable carrier or diluent.

28. The pharmaceutical composition of claim 27, wherein the pharmaceutical composition is in the form of a gel, optionally a hydrogel.

29. The pharmaceutical composition of claim 28, wherein said gel is selected from the group consisting of: Carbopol® 980, Carbopol® 974 and Carbopol® ETD 2020.

30. The pharmaceutical composition of any one of claims 27 to 29, wherein the concentration of methotrexate that is in the form bound to nanoparticle in said gel is in the range 0.5 mg/mL to 10 mg/mL, optionally about 2 mg/mL.

31. The pharmaceutical composition of any one of claims 27 to 30, wherein the nanoparticle core is of gold and the concentration of gold in said gel is in the range 1 mg/mL to 20 mg/mL, optionally about 4 mg/mL.

32. The pharmaceutical composition of any one of claims 27 to 31, wherein said composition is for topical administration.

33. The pharmaceutical composition of claim 27, wherein said composition is for systemic administration.

34. The nanoparticle of any one of claims 1 to 26 or a pharmaceutical composition of any one of claims 27 to 33 for use in medicine.

35. The nanoparticle of any one of claims 1 to 26 or a pharmaceutical composition of any one of claims 27 to 33 for use in the treatment of an inflammatory or autoimmune disorder in a mammalian subject.

36. The nanoparticle or composition for use according to claim 35, wherein said inflammatory or autoimmune disorder is selected from the group consisting of: psoriasis, psoriatic arthritis, scleroderma, rheumatoid arthritis, juvenile dermatomyositis, lupus, sarcoidosis, Crohn's disease, eczema and vasculitis.

37. The nanoparticle or composition for use according to claim 35, wherein said inflammatory or autoimmune disorder is a skin disorder.

38. The nanoparticle or composition for use according to claim 37, wherein said disorder is psoriasis.

39. The nanoparticle or composition for use according to any one of claims 35 to 38, wherein said nanoparticle or said composition is administered concurrently, sequentially or separately with a second anti-inflammatory agent.

40. The nanoparticle or composition for use according to claim 39, wherein said second anti-inflammatory agent comprises ciclosporin, hydroxycarbamide, dimethyl fumarate, a retinoid or biologic anti-inflammatory agent.

41. The nanoparticle or composition for use according to claim 40, wherein said biologic anti-inflammatory agent comprises an anti-TNFα antibody, an anti-TNFα decoy receptor, an anti-IL-17 antibody or an anti-IL-23 antibody.

42. A method of treating an inflammatory or autoimmune disorder in a mammalian subject, comprising administering a nanoparticle according to any one of claims 1 to 26 or a pharmaceutical composition according to any one of claims 27 to 33 to the subject in need of therapy.

43. The method of claim 42, wherein said inflammatory or autoimmune disorder is selected from the group consisting of: psoriasis, psoriatic arthritis, scleroderma, rheumatoid arthritis, juvenile dermatomyositis, lupus, sarcoidosis, Crohn's disease, eczema and vasculitis.

44. The method of claim 43, wherein said inflammatory or autoimmune disorder is a skin disorder.

45. The method of claim 44, wherein said disorder is psoriasis.

46. The method of any one of claims 42 to 45, wherein said nanoparticle or said composition is administered concurrently, sequentially or separately with a second anti-inflammatory agent.

47. The method of claim 46, wherein said second anti-inflammatory agent comprises ciclosporin, hydroxycarbamide, dimethyl fumarate, a retinoid or biologic anti-inflammatory agent.

48. The method of claim 47, wherein said biologic anti-inflammatory agent comprises an anti-TNFα antibody, an anti-TNFα decoy receptor, an anti-IL-17 antibody or an anti-IL-23 antibody.

49. Use of a nanoparticle according to any one of claims 1 to 26 or a pharmaceutical composition according to any one of claims 27 to 33 in the preparation of a medicament for use in a method according to any one of claims 36 to 42.

50. An article of manufacture comprising:
a nanoparticle according to any one of claims 1 to 26 or a pharmaceutical composition according to any one of claims 27 to 33;
a container for housing the nanoparticle or pharmaceutical composition; and
an insert or label.

51. The article of manufacture according to claim 50, wherein the insert and/or label provides instructions, dosage and/or administration information relating to the use of the nanoparticle or pharmaceutical composition in the treatment of an inflammatory or autoimmune disorder in a mammalian subject.
